(19) United States
(12) Patent Application Publication
(10) Pub. No.: US 2024/0252493 A1
(43) Pub. Date: Aug. 1, 2024

(54) Apelin Receptor Modulators for Treating Age-Related Muscle Conditions

(71) Applicant: BIOAGE LABS, INC., Richmond, CA (US)

(72) Inventors: Kristen Patricia Fortney, Derry, NH (US); Eric Kim Morgen, Toronto (CA); Justin Rebo, Derry, NH (US); Robert Hughes, Berkeley, CA (US); Fred Aswad, Berkeley, CA (US); Peng Khun Leong, San Francisco, CA (US); Sashanaz H. Igdari, San Ramon, CA (US); Paul David Rubin, Henderson, NV (US)

(21) Appl. No.: 18/286,070

(22) PCT Filed: Apr. 6, 2022
(86) PCT No.: PCT/US2022/023732

§ 371 Date: Oct. 6, 2023

Related U.S. Application Data
(60) Provisional application No. 63/171,475, filed on Apr. 6, 2021.
(60) Provisional application No. 63/272,419, filed on Oct. 27, 2021.

(51) Int. Cl. A61K 31/506; A61K 9/00; A61P 21/00

(52) U.S. Cl. CPC A61K 31/506; A61P 21/00; A61K 9/0053

(57) ABSTRACT

Apelin receptor modulators can improve physical performance, slow progression of age-related frailty, and can reduce age-related muscle weakness in human patients. This disclosure provides methods for treating muscle conditions using a particular class of apelin receptor modulators (e.g., agonists). The muscle condition can be an age-related muscle condition. Also provided is a method for maintaining and/or increasing muscle mass and/or muscle strength in an elderly subject by administration of the apelin receptor modulator. In some embodiments, the apelin receptor modulator (e.g., agonist) is BGE-105, or a pharmaceutically acceptable salt thereof.

### 1. CROSS-REFERENCE TO RELATED APPLICATIONS

This application claims the benefit of U.S. Provisional Application Nos. 63/171,475, filed Apr. 6, 2021, and 63/272,419, filed Oct. 27, 2021, the disclosures of which are hereby incorporated in their entirety by reference.

### 2. BACKGROUND

As people age, they accumulate physiologic and pathophysiologic changes; these accumulated age-related changes predispose a person to death from various external and internal stressors. Frailty is highly prevalent in old age and considered synonymous with disability, comorbidity, and other characteristics that confer high risk for falls, disability, nursing home admission, hospitalization, and mortality. Frailty is considered a clinical syndrome which can be characterized according to indices of frailty that are composite measures of such age-related changes. As the median age of the population increases, there is an increasing need for drugs that reduce or counteract the accumulation of age-related deficits including frailty in elderly individuals.

### 3. SUMMARY

This disclosure provides methods for treating muscle conditions using a particular class of apelin receptor modulators, and in particular treatment for a variety of age-related muscle conditions. In some embodiments, the apelin receptor modulator is an apelin receptor agonist.

We applied bioinformatic and machine learning approaches to analyze human data using survival predictor models and discovered an association of apelin protein levels with future aging outcomes. We discovered that higher circulating levels of apelin are associated with reduced all-cause mortality (p-0.0002)—that is, greater longevity. In addition, our analyses demonstrated that higher levels of apelin are associated with better future physical function, and measures of frailty.

Based on this discovery, we tested a modulator of the apelin receptor, BGE-105, for its effect on aged mice in models of frailty. BGE-105 has the structure shown below:

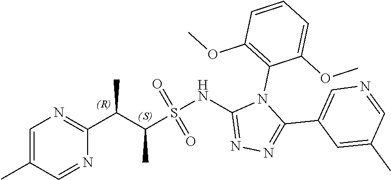

BGE-105 (also referred to as AMG-986) is known to activate the apelin receptor and induces a cardiovascular response in rats (Ason et al., JCI Insight. 5 (8):1-16 (2020)). Clinical trials were performed with AMG-986 to study the safety, tolerability, and pharmacokinetics in healthy subjects and heart failure subjects (NCT03276728) those with impaired renal function (NCT03318809). Nevertheless, the compound's effect on muscle loss and function in elderly individuals is unknown.

In a first set of experiments, we demonstrated that aged mice (24-month-old) treated with BGE-105 exhibit a statistically significant increase in voluntary motor activity (p=0.00228) and a statistically significant improvement in grip strength (p=0.04) as compared to age-matched controls, indicating improved physical health and increased muscle strength.

In addition, aged mice (18-month-old) first injected with a cardiotoxin and then treated with BGE-105 showed significantly higher levels of several mRNA transcripts which are indicative of muscle regeneration.

Third, immortalized muscle precursor cells from human patients showed a dose-dependent relationship between cell growth and differentiation, and concentration of BGE-105.

Lastly, immobilized aged mice (20-months-old) that were orally dosed with BGE-105 displayed significantly reduced muscle atrophy as compared to immobilized mice that were injected with vehicle.

Thus, an apelin receptor modulator can increase physical performance, counteract age-related frailty, and can reduce age-related muscle weakness.

Accordingly, a first aspect of the present disclosure provides a method for treating a muscle condition in a subject, the method including administering to a subject in need thereof an effective dose of an apelin receptor modulator. In some aspects of the invention the modulator is an apelin receptor agonist, such as an apelin receptor agonist of formula (I) or (II) as described herein. In some embodiments, the muscle condition is an age-related muscle condition. In some embodiments, the apelin receptor agonist is BGE-105, or a pharmaceutically acceptable salt thereof.

In another aspect, the present disclosure provides a method for maintaining and/or increasing muscle mass and/or muscle strength in an elderly subject, the method comprising administering to a subject in need thereof an effective dose of an apelin receptor agonist, such as an apelin receptor agonist of formula (I) or (II) as described herein. In some embodiments, the apelin receptor agonist is BGE-105, or a pharmaceutically acceptable salt thereof.

In some embodiments of the methods of this disclosure, the subject is human and has, or is identified as having, one or more of low muscle strength, low muscle force, low muscle mass, low muscle volume. In some embodiments, the muscle is skeletal muscle. In some embodiments, the muscle is the diaphragm, tibialis anterior, tibialis posterior, gastrocnemius, sartorius, vastus intermedius, vastus laterals, vastus medialis, soleus, or extensor digitorum longus. In some embodiments, the muscle is diaphragm muscle.

In some embodiments of the methods of this disclosure, the subject is human and has, or is identified as having, one or more of diabetes mellitus, insulin insensitivity, cardiovascular disease, and neurologic disease.

In some embodiments of the methods of this disclosure, the subject is human and has low muscle strength, low muscle force, low muscle mass, and/or low muscle volume due to disuse atrophy after immobilization.

In some embodiments of the methods of this disclosure, the subject is human and has diaphragm dysfunction or diaphragm atrophy.

### 4. BRIEF DESCRIPTION OF THE DRAWINGS

These and other features, aspects, and advantages of the present invention will become better understood with regard to the following description, and accompanying drawings, where:

FIG. 1 shows the structure of BGE-105.

FIGS. 2A-2D graph results from a bioinformatic survival model examining the relationship between serum levels of a given protein and future risk of all-cause mortality (i.e. longevity) or retaining full mobility in human healthy aging cohorts, using non-public clinical outcome data and proteomics data generated on archived samples. FIG. 2A shows a Kaplan-Meier curve of survival probability for humans in the top 20% (blue) versus bottom 20% (red) of apelin protein levels, demonstrating that in humans higher circulating levels of apelin are associated with decreased all-cause mortality (p=0.0002). FIG. 2B shows a similar model of full mobility by protein levels, where higher circulating levels of apelin are associated with increased retention of full mobility (p=0.0082). The hazard ratio for apelin was 0.88 in FIG. 2A and 0.89 in FIG. 2B. In both cases, the hazard ratio given is for the continuous Cox proportional hazards analysis, which is fitting to the entire distribution of apelin measurements. P-values in FIGS. 2A and 2B are calculated for these hazard ratios, based on testing the null hypothesis that the hazard ratio in each case equals 1. FIG. 2C, shows the serum abundance of the apelin protein module (highlighted by the green oval) in the Honolulu Heart Study (HHS) cohort. Each node represents a protein, and the edges between the nodes represent significant correlations. FIG. 2D shows the first principal component of the apelin protein module and death rate. The relative death rate (log; y-axis) was derived from the multivariate Cox regression model for the first principal component (PC1) after adjusting for age, smoking pack years, and alcohol status. The reference used was the median value of PC1.

FIGS. 3A-3I show the effect of BGE-105 on activity and muscle strength of 24-month-old C57BL/6 mice. FIGS. 3A and 3D show the results of repeated experiments on C57BL/6 mice on an activity wheel in their cages, with the readout being km/day. In FIGS. 3A and 3D the red dots, and associated line, are for mice that were treated with BGE-105, whereas the green dots, and associated line, are for mice that were not treated with BGE-105. Kendall rank correlation tau p=0.00228 and 1.14e−04, respectively. In FIGS. 3B and 3C the black dots are for mice that were treated with BGE-105, whereas the red dots are for mice that were treated with vehicle only, it shows that on average there was an increase in the latency to fall in the grid hang test (a measure of increased muscle strength) with mice that were treated with BGE-105 as opposed to vehicle. At the end of the study, the BGE-105-treated mice had significantly higher tissue wet weight in the tibialis anterior (TA) (FIG. 3F), and a trend toward higher tissue wet weight in the gastrocnemius and quadriceps (FIGS. 3G and 3H), and no difference in heart (FIG. 3I). The BGE-105-treated mice also had higher body weights (FIG. 3E), although the increase was not significant, just at p-value cutoff.

FIGS. 4A-4D depict increased levels of pAMPK (FIG. 4A-4B) and pAkt (FIGS. 4C-4D) in BGE-105-treated mice vs. vehicle-treated mice.

FIGS. 4E-4F depict that per unit mass, the soleus contained about half as much APLNR receptor as the heart, potentially explaining the stronger response in heart tissue.

FIGS. 5A-5B depict the levels of apelin receptor protein found in rat tissue. FIGS. 5C-5D depict oral dosing of rats with BGE-105 for 5 consecutive days induced phosphorylation of Akt in the TA in a dose-dependent manner, with 50 mg/kg BID eliciting the strongest response. FIGS. 5E-5F depict the same for Erk. FIGS. 5G-5I depict the effect of chronic administration of BGE-105 on apelin receptor protein levels in the TA.

FIGS. 6A-6B show that in cells stably expressing human apelin receptor, BGE-105 was 10-fold more potent than Pyr¹-Apelin-13 and in cells stably expressing mouse apelin receptor, BGE-105 was 30-fold more potent than Pyr¹-Apelin-13.

FIGS. 7A-7F show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the tibialis anterior of aged (18-month-old) mice either 3 days or 7 days post injection of cardiotoxin. FIG. 7A shows a significant increase in Pax7 levels for apelin (injection) and both BGE-105 (P.O.) dosages 7 days post administration. FIG. 7B shows a significant increase in the levels of MyoD at both 3 and 7 days post injection for apelin and both BGE-105 dosages. FIG. 7C shows a significant increase in MyoG levels 7 days post administration for apelin and both BGE-105 dosages. FIG. 7D shows a significant increase in MyHC3 levels 7 days post injection for apelin and BGE-105 (P.O.) at both dosages. FIG. 7E shows a significant change in the MyHC8 levels 7 days post injection for apelin and both BGE-105 dosages. FIG. 7F shows a significant change in the Myf5 levels 7 days post injection for apelin and both BGE-105 dosages.

FIGS. 7G-7L show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the gastrocnemius of aged (18-month-old) mice either 3 days or 7 days post injection with cardiotoxin. FIG. 7G shows no significant change in Pax7 levels. FIG. 7H shows a significant increase in the levels of MyoD at both 3 and 7 days post injection for apelin and both BGE-105 dosages. FIG. 7I shows no significant change in MyoG levels. FIG. 7J shows a significant increase in MyHC3 levels only at 7 days post injection for the larger BGE-105 injection. FIG. 7K does not show a change in the MyHC8 levels at any time point for any injection. FIG. 7L shows no significant change in Myf5 levels.

FIGS. 7M-7R show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the tibialis of young (3-month-old) mice either 3 days or 7 days post injection with cardiotoxin. FIG. 7M shows no change in Pax7 levels. FIG. 7N shows no significant increase in the MyoD levels. FIG. 7O shows no difference in MyoG levels. FIG. 7P shows no significant change in the MyHC3 levels. FIG. 7Q shows no significant change in the MyHC8 levels. FIG. 7R shows no difference in Myf5 levels.

FIGS. 7S-7X show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the gastrocnemius of young (3-month-old) mice either 3 days or 7 days post injection with cardiotoxin. FIG. 7S shows no change in Pax7 levels. FIG. 7T shows no change in the levels of MyoD. FIG. 7U shows no change in MyoG levels. FIG. 7V shows an increase in MyHC3 levels only 3 days post injections for the smaller BGE-105 dosage. FIG. 7W shows a non-significant increase in MyHC8 levels 3 days post injections for apelin and both BGE-105 dosages. FIG. 7X shows no change in Myf5 levels.

FIGS. 7Y-7Z show the cross-sectional area of the tibialis at day 3 and day 7 post injection of cardiotoxin after treatment with PBS, Pyr¹-Apelin-13 (apelin), BA1 (BGE-105 50 mg/kg/d), and BA2 (BGE-105 200 mg/kg/d). FIG. 7Y shows representative histological cross-sectional slices of the tibialis 3 and 7 days post injection with treatment of PBS, apelin, BA1, or BA2. FIG. 7Z shows the quantification of the cross-sectional histological slides, which shows a significant increase in cross sectional area for apelin, BA1, and BA2 at both 3 and 7 days post injection.

FIGS. 7AA-7BB show the amount of centrally nucleated fibers (CNM) as part of the regenerative process after cardiotoxin injection for PBS, Pyr¹-Apelin-13 (apelin), BA1 (BGE-105 50 mg/kg/day), and BA2 (BGE-105 200 mg/kg/day) treatments. Mice were 18-months old. FIG. 7AA shows representative distribution of DAPI stained nuclei and positively-stained eMHC fibers. FIG. 7BB shows the quantification of the amount of centrally nucleated myofibers (CNM). There was a significant increase in the amount of CNM after apelin and the higher of the two BGE-105 treatments.

FIGS. 8A-8C show the ability of BGE-105 to increase the proliferation of immortalized human muscle cells from both younger (25-years-old) and older (79-years-old) subjects. FIG. 8A shows the experimental protocol with an initial incubation (Treatment #1) during in vitro proliferation of cells (days 0-4) with a second incubation (Treatment #2) during the differentiation stage into myotubes (days 4-18). The treatments were DMSO (0.1%), Pyr¹-Apelin-13 (1 nM), or BA (BGE-105) at 0.05, 0.5, 5, or 50 nM. FIG. 8B shows the proliferation of the cells (measured at day 4) from the younger subject with an increase at 5 nM and a significant increase at 50 nM of BGE-105 treatment. FIG. 8C shows the proliferation of the cells from the older subject with a significant increase at 5 nM of BGE-105 and an increase at 50 nM of BGE-105 treatment.

FIGS. 8D-8K show the levels of Pax7, MyoD, MyoG and Myf5 expression in immortalized muscle cells from older (79-years old) and younger (25-years-old) subjects after incubation with DMSO (0.1%), Pyr¹-Apelin-13 (apelin) (1 nM), or BGE-105 at 0.05, 0.5, 5, or 50 nM. FIG. 8D shows the levels of Pax7 in the younger cells. BGE-105 at 5 and at 50 nM recapitulated the levels of apelin. FIG. 8E shows the levels of MyOD expression levels after treatment in the younger cells. There was a very significant increase of MyoD expression at 5 nM of BGE-105. FIG. 8F shows the MyoG expression after treatment in the younger cells. There was no change in the level of Myf5 relative to the control for any amount of BGE-105. FIG. 8G shows the levels of Myf5 expression after treatment in the younger cells. At 5 and 50 nM the levels were equal to apelin treatment. FIG. 8H shows Pax7 levels after treatment in older cells. There was a significant increase at all treatment doses as compared to control. FIG. 8I shows the levels of MyOD after treatment of cells derived from the older donor. There was an increase at all treatment levels with the higher dosages, approaching the levels of expression caused by apelin. FIG. 8J shows the levels of MyoG after treatment in the older cells. There was an increase at all treatment levels, approaching the levels of expression caused by apelin. FIG. 8K shows the levels of Myf5 expression after treatment in cells derived from the older donor. At 5 and 50 nM the levels were equal to apelin treatment.

FIGS. 9A-9M show effects of BGE-105 in preventing disuse-induced muscle atrophy in aged mice. See Example 7.

### 5. DETAILED DESCRIPTION OF THE INVENTION

### 5.1. Apelin Receptor Modulators and Frailty

### 5.1.1. Survival Predictability Model

The present disclosure describes a bioinformatics model that generally relates to building of survival predictor models that output a survival metric. Such survival metrics may relate to survival related observables, such as survival expectancy and/or risk of death. Survival predictor models may be built by selecting observables that relate to survival periods (“aging indicator”). Such aging indicators may comprise variables that correlate with all-cause mortality, such as certain clinical factors. Survival predictor models can utilize one or a plurality of survival biomarkers together with one or more aging indicators to generate a survival metric.

In some embodiments, a survival predictor model of the present disclosure examines the relationship between serum levels of apelin, and future risk of all-cause mortality in human healthy aging cohorts, with clinical outcome data proprietary to those cohorts and proteomics data generated on archived samples, based on survival modeling. Additionally, the relationship between apelin and mobility decline events (e.g., a decrease in ability of walking, stair-climbing, or transferring activities as shown by self-reported difficulty of these activities) is examined using a Cox proportional hazards model, with a hazard ratio and associated p-value generated for apelin.

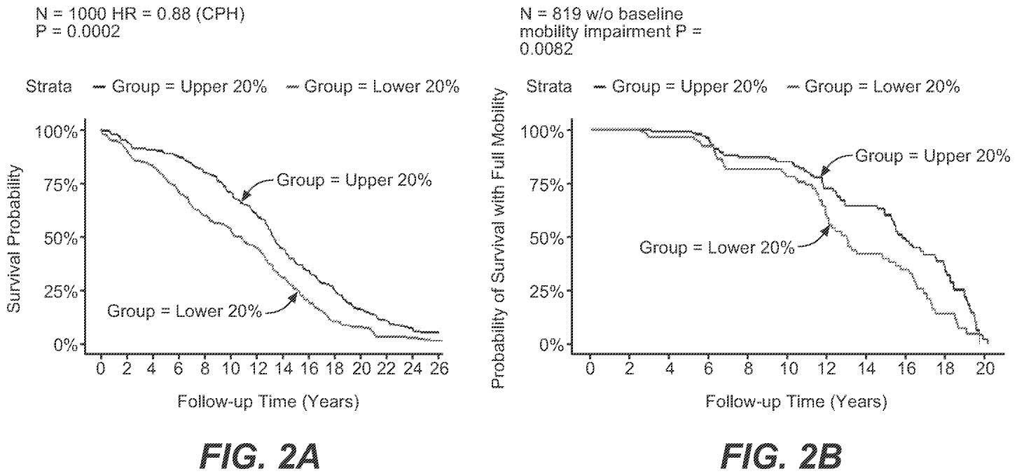

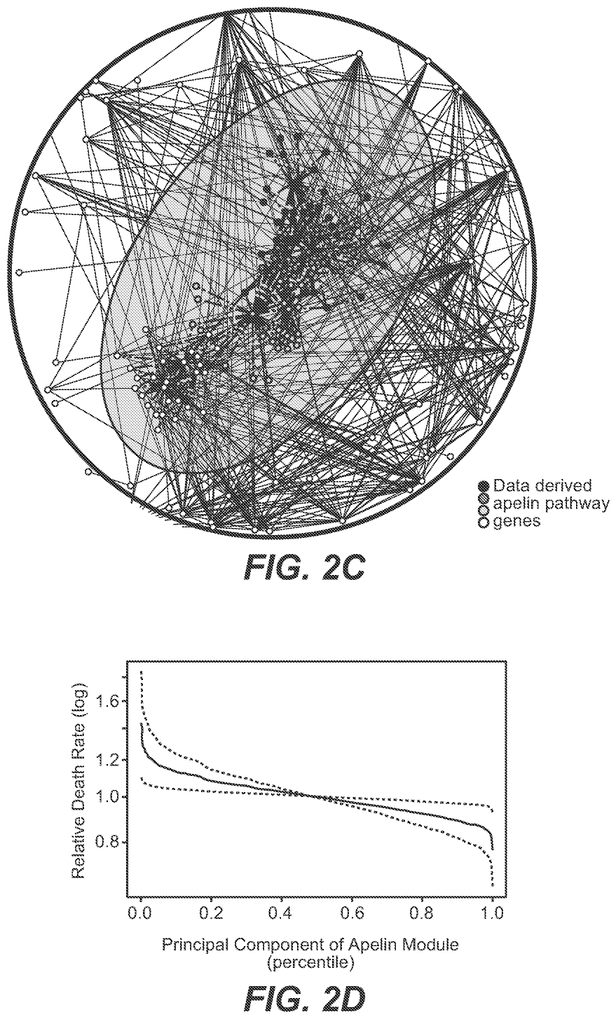

We applied such bioinformatic and machine learning approaches to analyze human data using survival predictor models and discovered an association of apelin receptor levels with future aging outcomes. We discovered that higher circulating levels of apelin are associated with decreased all-cause mortality (p=0.0002)—that is, greater longevity. See, e.g., FIG. 2A. In addition, our analyses demonstrated that higher levels of apelin are associated with better future physical function in healthy aging human subjects. FIG. 2B shows a similar model that indicates higher circulating levels of apelin are associated with increased retention of full mobility (p=0.0082).

### 5.1.2. Apelin Receptor Expression in Aged Subjects

There is also a demonstrated relationship between the age of mice or humans and apelin receptor expression. The expression of apelin receptor decreases with age in skeletal muscle. Samples taken from frail older patients showed an even larger decrease in apelin receptor levels. Further details are provided in the experimental section, see, e.g., Example 1 and FIG. 2.

### 5.1.3. Aged Mouse Study

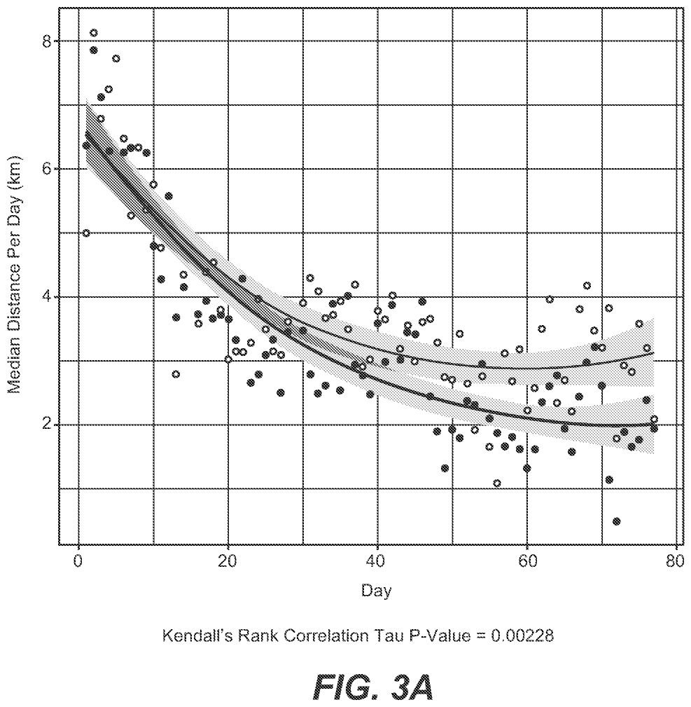

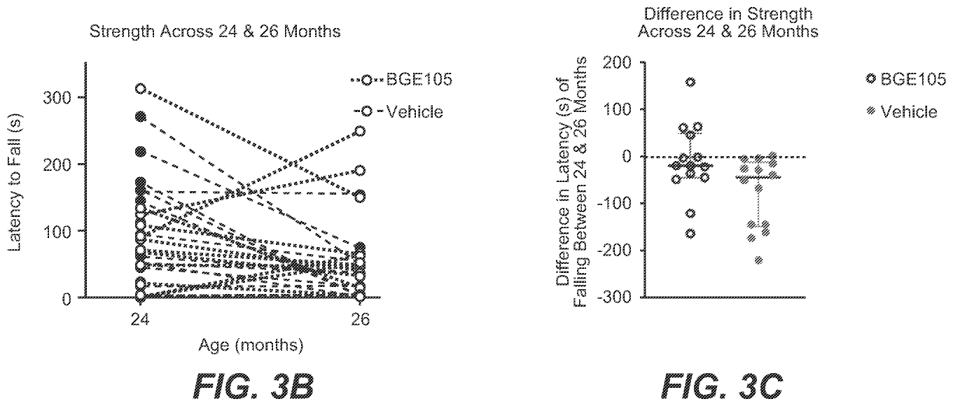

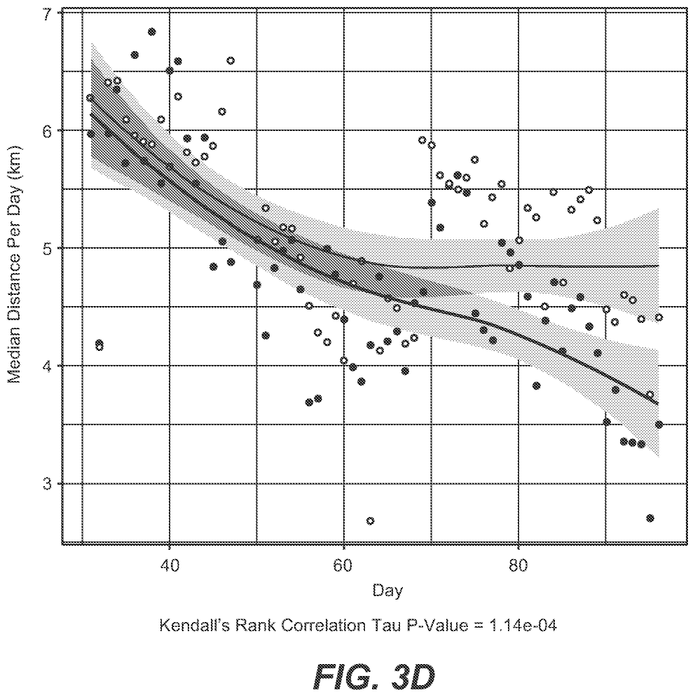

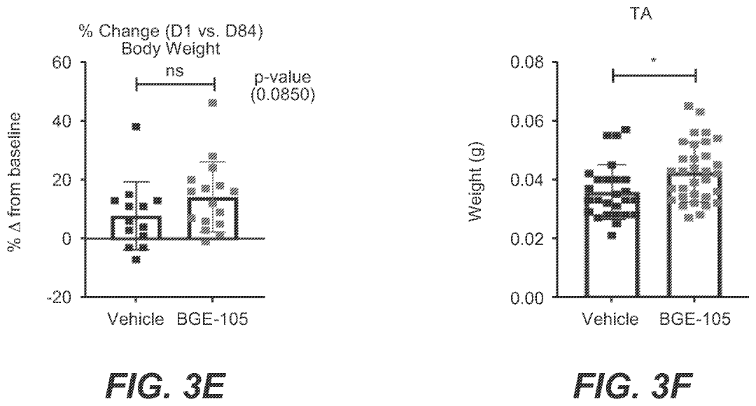

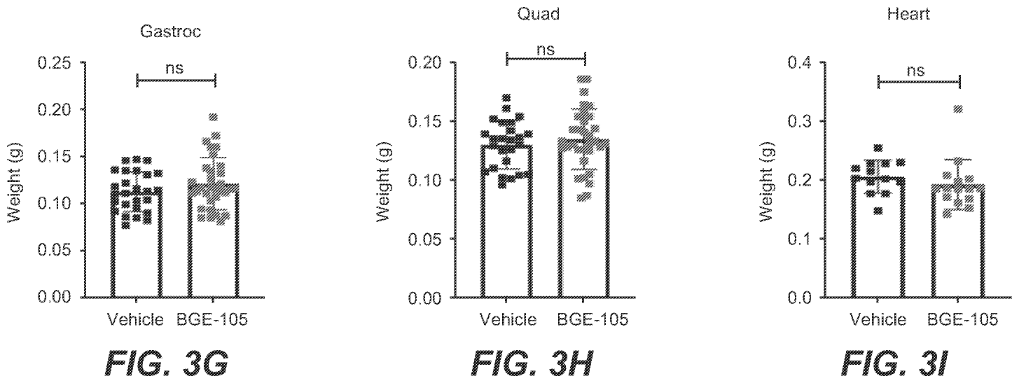

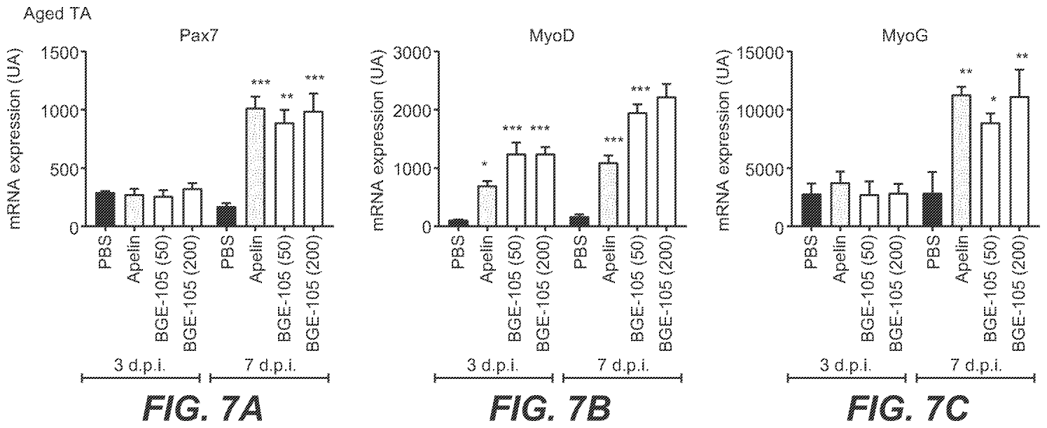

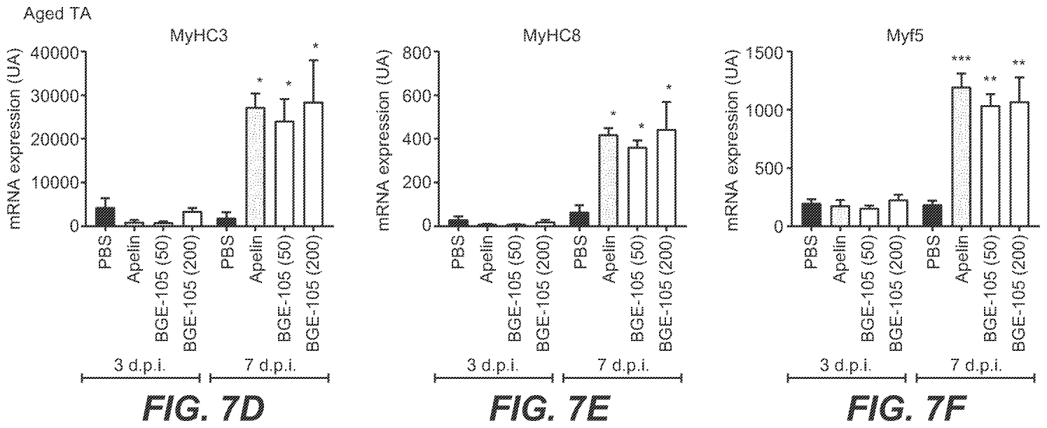

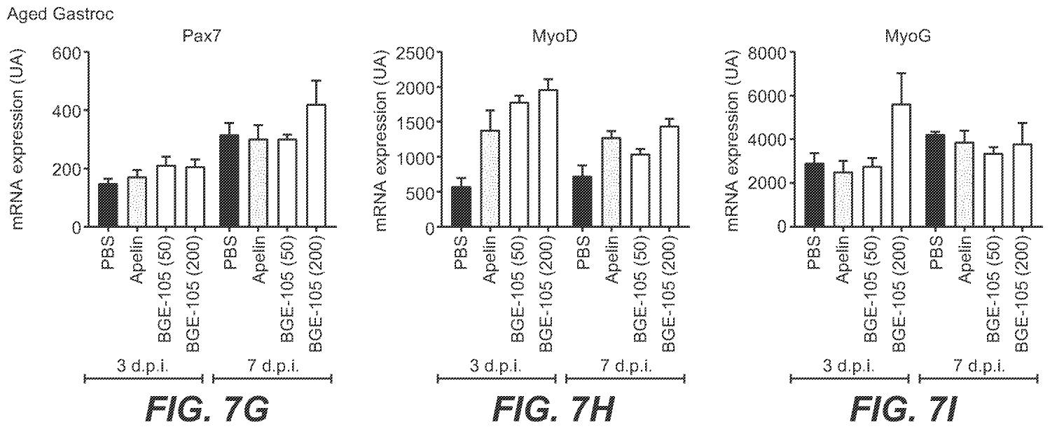

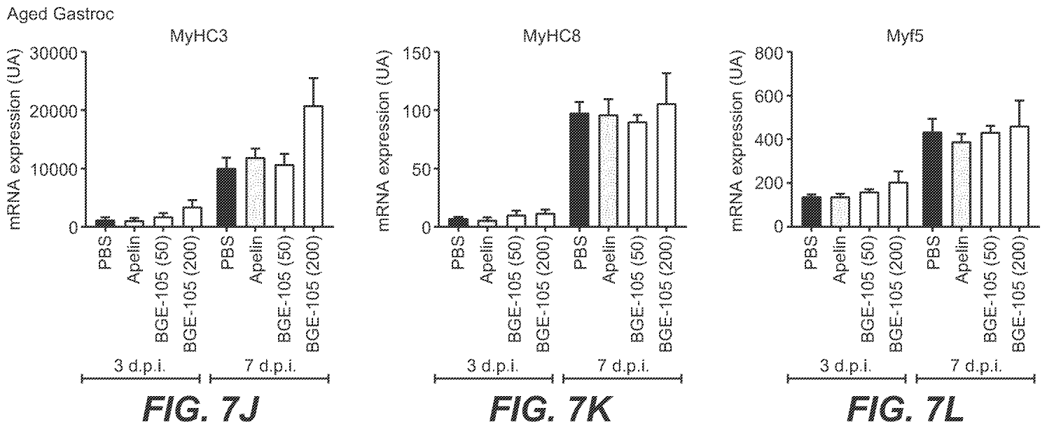

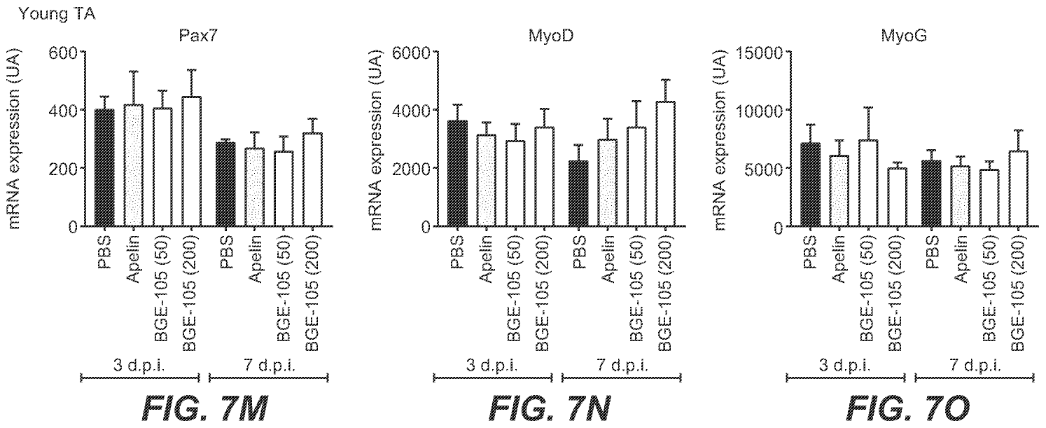

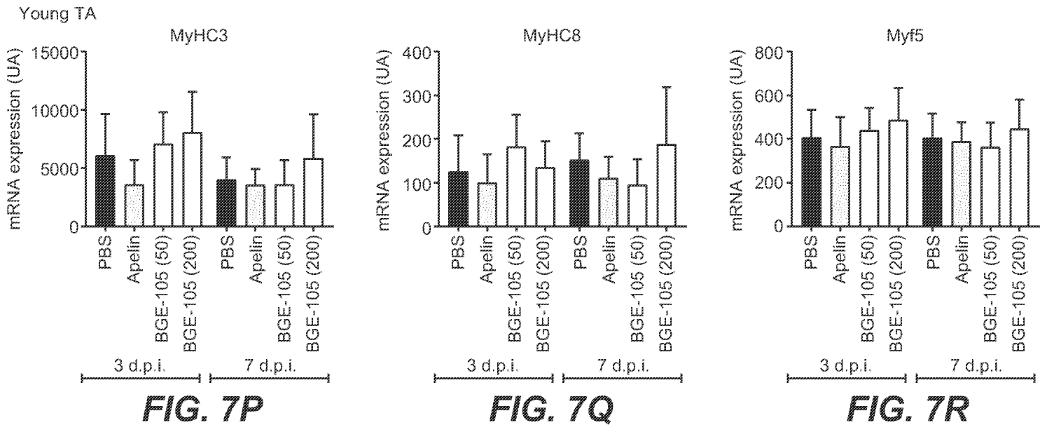

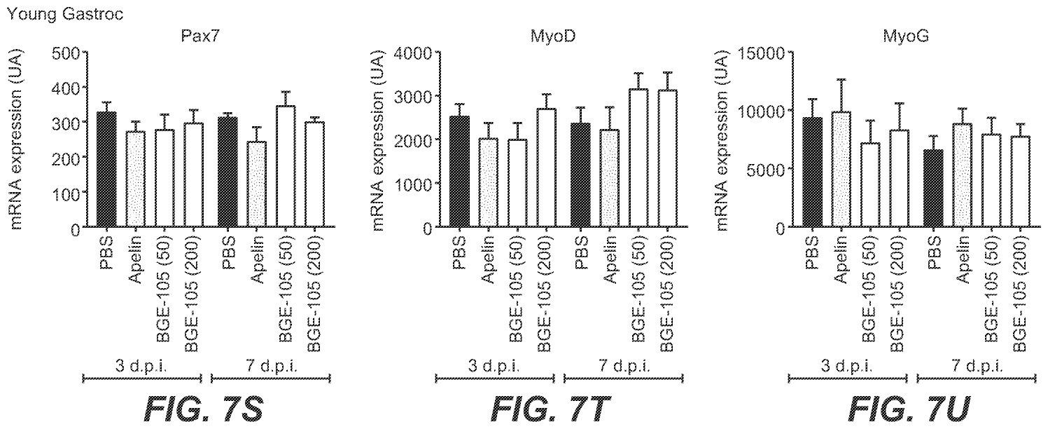

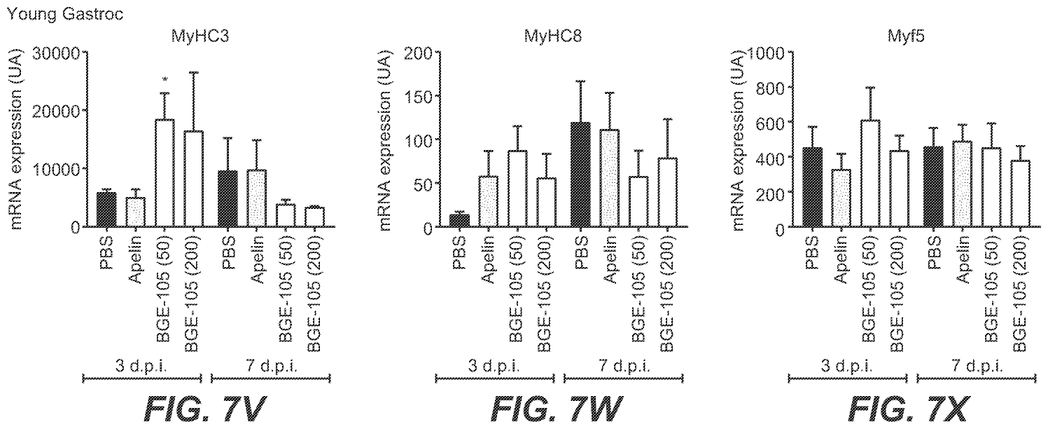

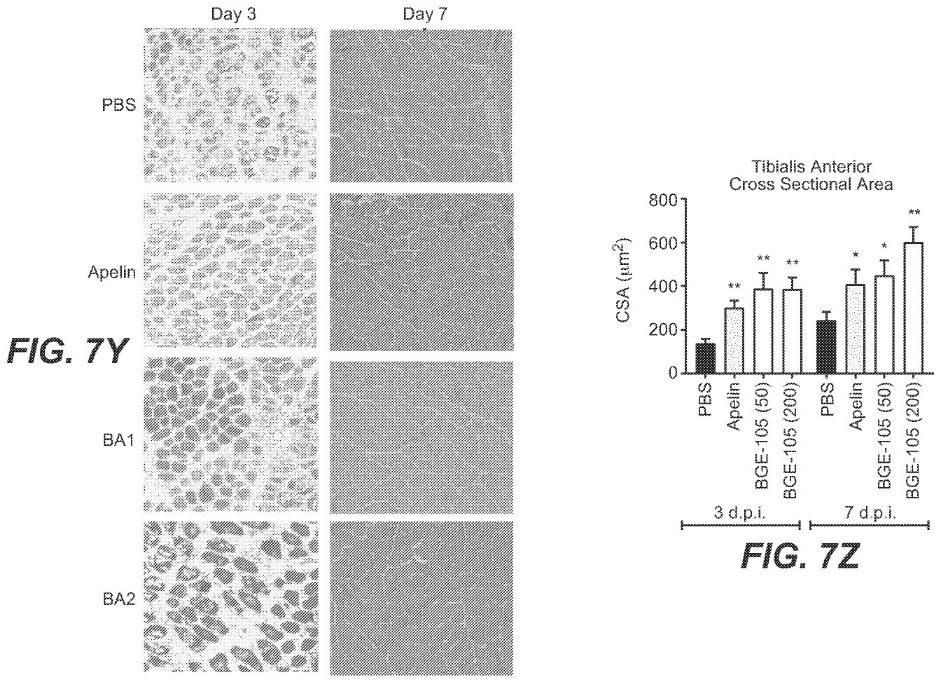

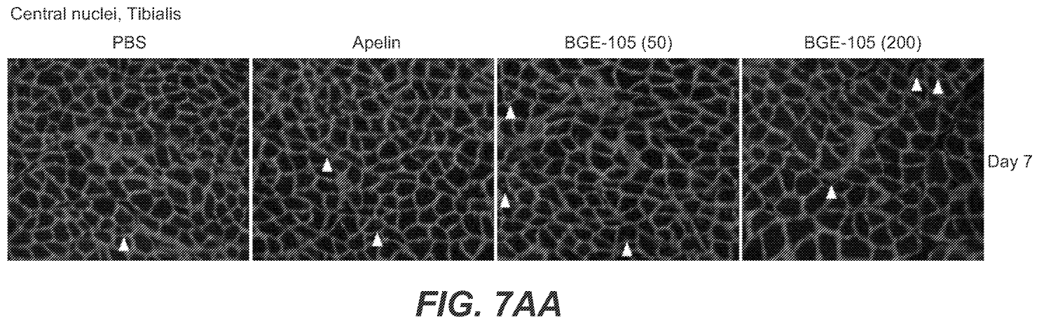

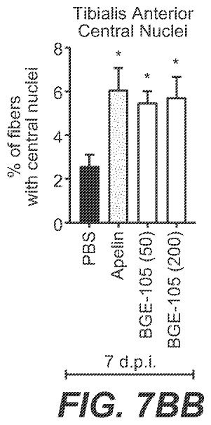

We demonstrated that aged mice (24-months old) treated with BGE-105 exhibit a statistically significant increase in voluntary activity (p=0.002) and an improvement in grip strength (p=0.04) as compared to age-matched controls, indicating improved physical health and increased muscle strength (FIGS. 3A-3I). In addition, aged mice (18-months old) first injected with a cardiotoxin and then treated with BGE-105 showed significantly higher levels of several transcripts which are indicative of muscle regeneration (FIGS. 7A-7X). Cells from these mice were frozen and histological sections were stained. The sections showed a dose-dependent increase in central nucleated fibers (indicative of muscle growth) (FIGS. 7AA-7BB). Further details are provided in the experimental section, see, e.g., Examples 2-5 and FIGS. 3-7.

### 5.1.4. BGE-105 Activates the Apelin Pathway in Vitro

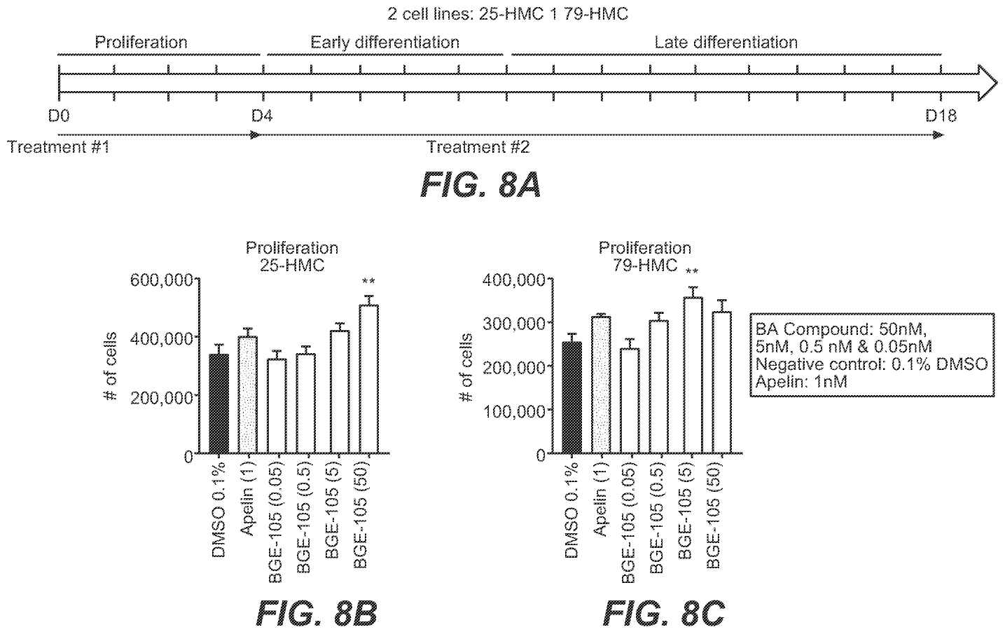

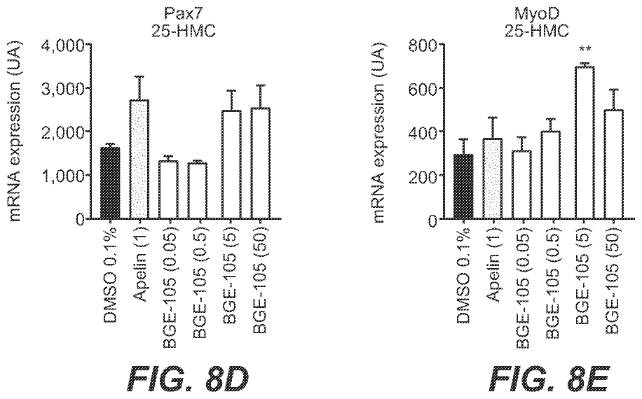

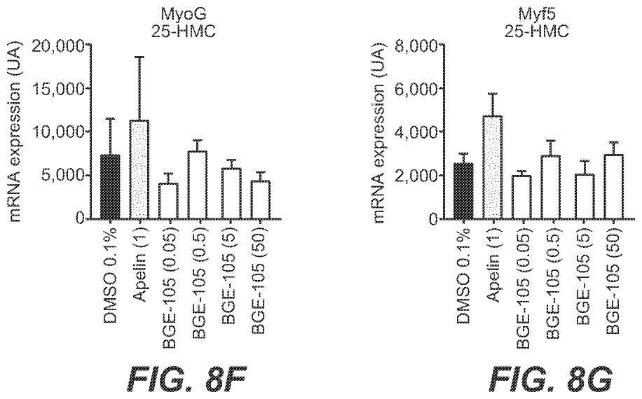

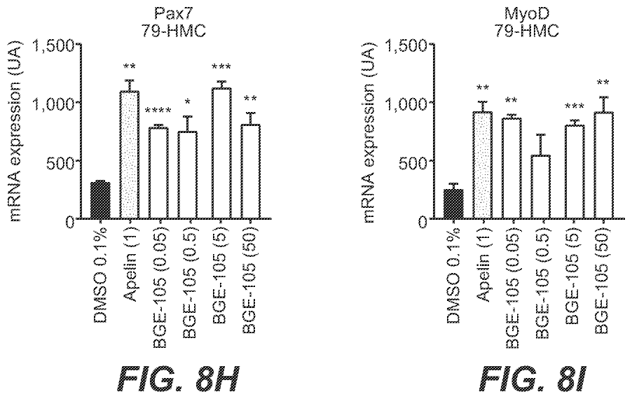

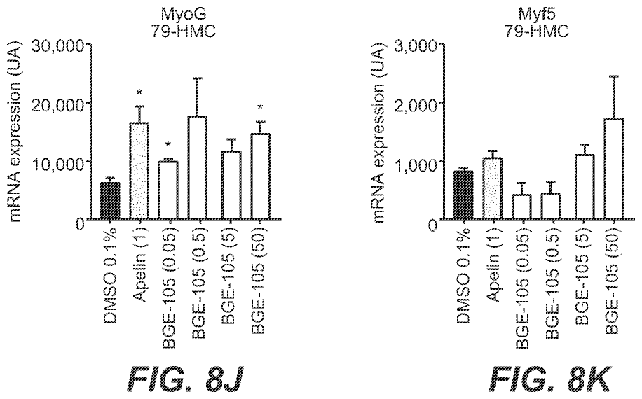

We demonstrated that immortalized human muscles from younger and older patients showed increased proliferation after treatment with increased dosages of BGE-105 (FIGS. 8B-8C). The younger cells showed a significant increase in cell proliferation at 50 nM, and the older cells had a significant increase in cell proliferation at 5 nM. Further details are provided in the experimental section, see, e.g., Example 6 and FIG. 8.

### 5.1.5. BGE-105 Prevents Atrophy in Immobilized Mouse Muscle

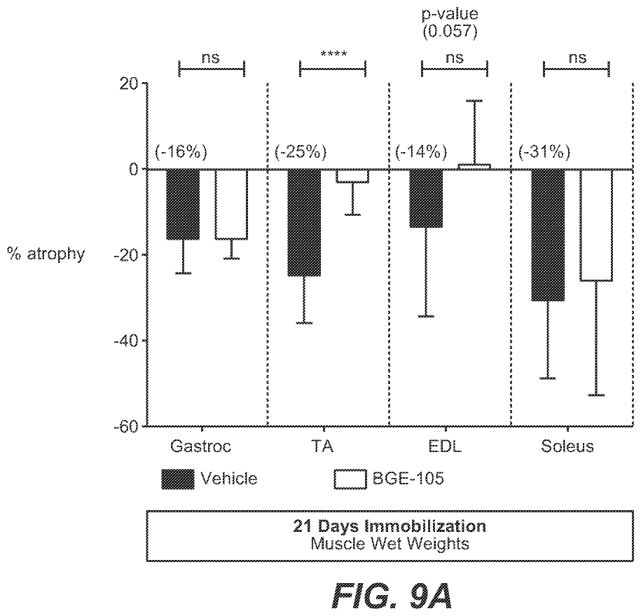

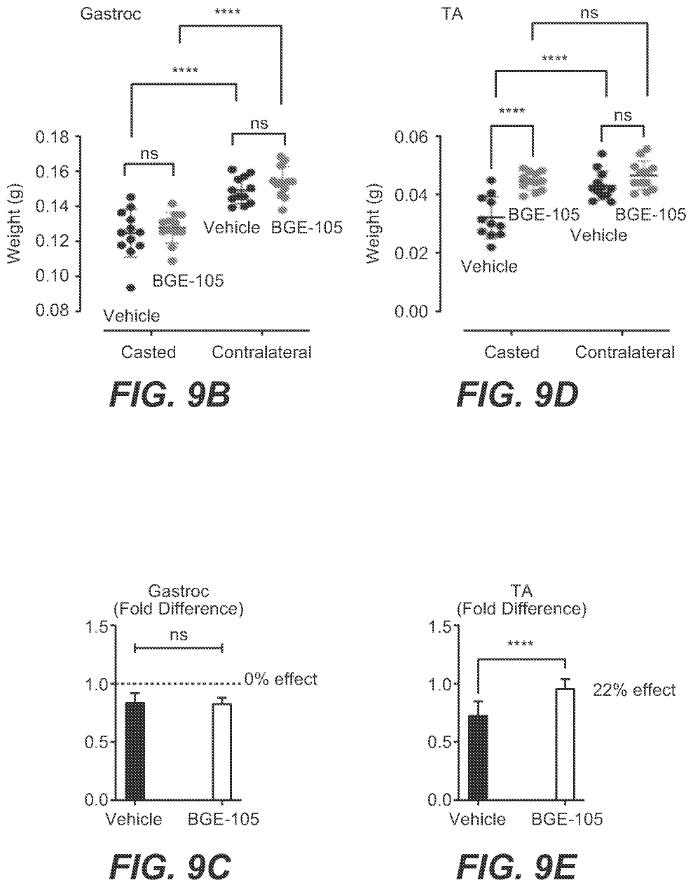

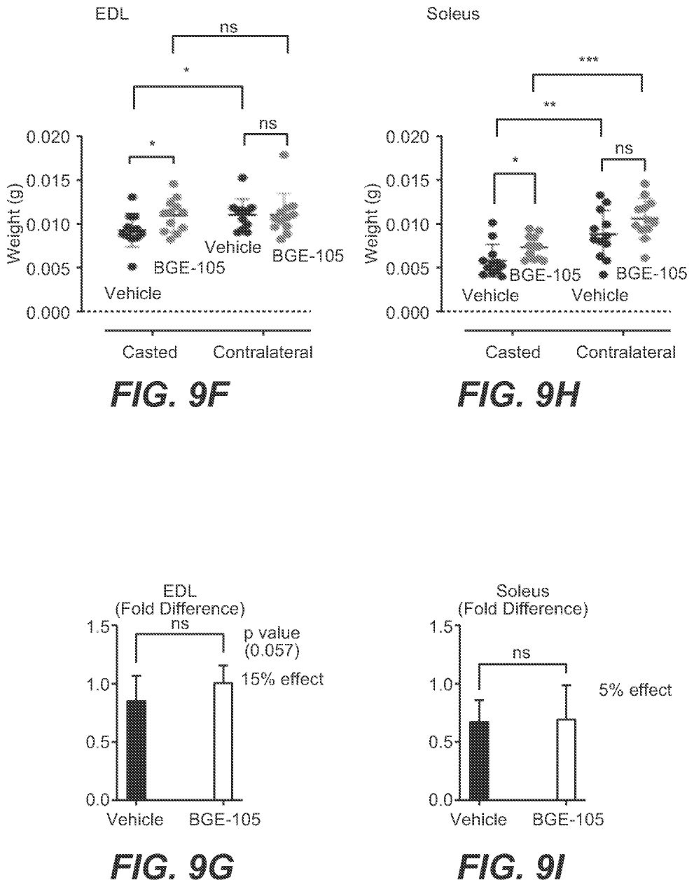

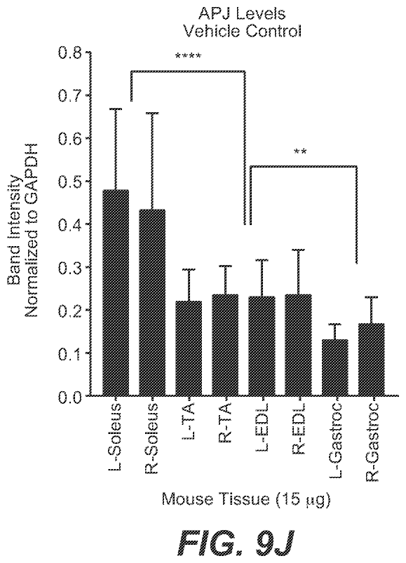

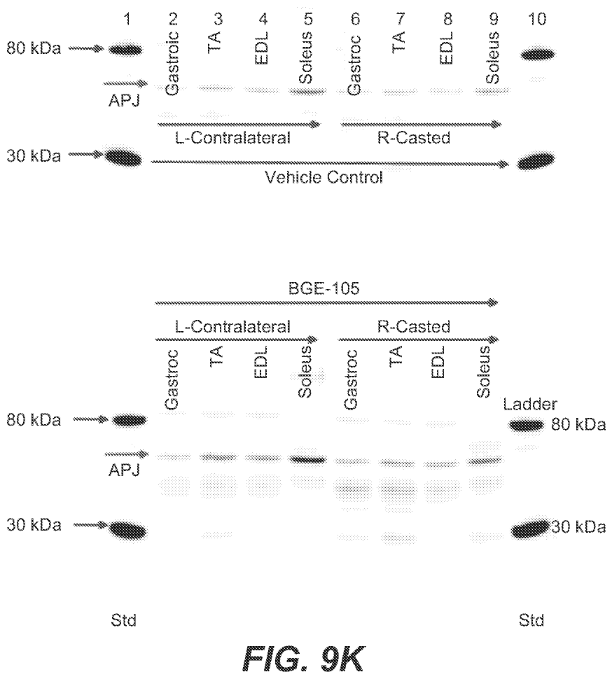

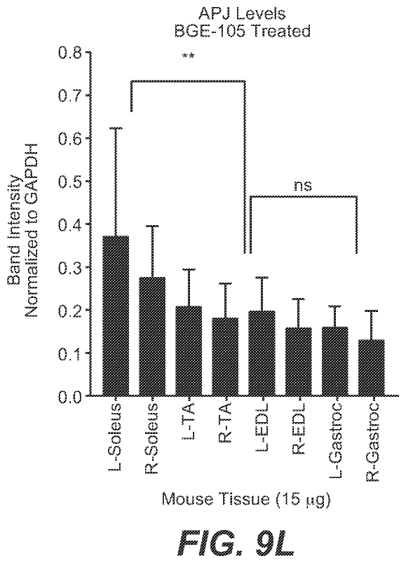

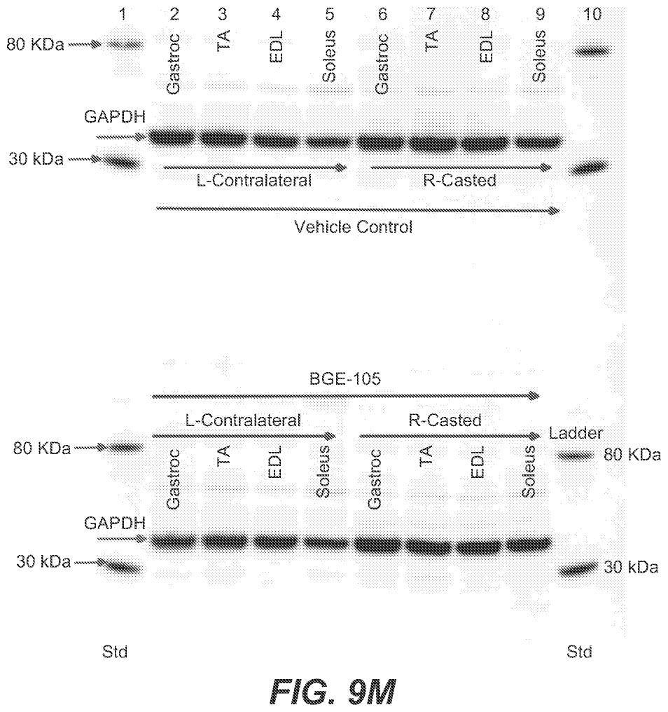

We demonstrated that 20-month-old mice which were immobilized and treated with BGE-105 showed a significant improvement in maintaining muscle weight in the tibialis anterior as compared to vehicle-treated controls. (FIGS. 9D and 9E). There was also a near significant improvement in muscle weight as compared to vehicle in the extensor digitorum longus (FIGS. 9F and 9G) and in the soleus (FIGS. 9H and 9I) muscle as compared to vehicle-treated controls.

There was a significant decrease in the percent atrophy in the tibialis anterior muscle, a near significant decrease in the percent atrophy in the extensor digitorum longus, a marginal improvement in the percent atrophy in the soleus, and no improvement in the gastrocnemius (FIG. 9A). Further details are provided in the experimental section, see, e.g., Examples 7 and FIG. 9.

### 5.2. Methods of Treating Age-Related Muscle Conditions

Accordingly, in a first aspect the present disclosure provides a method of treating a subject for a muscle condition, such as a muscle condition associated with aging, using an apelin receptor modulators. The method includes administering to a subject a therapeutically effective amount of an apelin receptor modulator of formula (I) or (II) (e.g., as described herein).

The “muscle condition associated with aging” (referred to interchangeably herein as an “age-related muscle condition”) refers to a degenerative disease or condition or impairment associated with muscle in a mammalian subject. In some embodiments, the muscle is skeletal muscle. Skeletal muscle is considered an organ of the muscular system. Skeletal muscle can include muscle tissues responsible for skeletal movement. For example, skeletal muscle can include muscles under conscious or voluntary control, such as striated muscles.

In some embodiments, other parts of the mammal can be affected by an age-related muscle condition, such as blood vessels (e.g, arteries), nerves, bones, or skin. In some embodiments, the age-related muscle condition is associated with inflammation or impairment of mitochondrial function.

Examples of muscle conditions that can be targeted for treatment according to the methods of this disclosure include, but are not limited to, sarcopenia, frailty, muscle weakness due to hip fracture, reduction in risk of hip fracture, ICU associated muscle weakness, muscle atrophy, diaphragm disfunction, diaphragm atrophy, ventilator-induced diaphragmatic dysfunction (VIDD), immobilization associated muscle weakness, immobility associated muscle weakness, recovery from muscle injury, and muscle wasting. In certain embodiments, the muscle condition is acute muscle atrophy. In some embodiments, the patient that has the muscle condition is on bedrest. In certain embodiments, the muscle condition is chronic muscle loss. In certain embodiments, the muscle condition is ICU diaphragm atrophy.

In some embodiments, the muscle condition is sarcopenia. Sarcopenia is a condition characterized by loss of skeletal muscle mass and function. When this condition is associated with aging, it can also be referred to as age-related sarcopenia. Diagnosis of sarcopenia can be achieved via an assessment of low muscle mass plus the presence of low muscle function (low muscle strength/weakness or low physical performance) (see e.g., Cruz-Jentoft et al., (2010) Sarcopenia: European consensus on definition and diagnosis Report of the European Working Group on Sarcopenia in Older People. Age and Ageing; 39: 412-423; Muscaritoli et al., (2010) Consensus definition of sarcopenia, cachexia and pre-cachexia: joint document elaborated by Special Interest Groups (SIG) “cachexia- anorexia in chronic wasting diseases” and “nutrition in geriatrics”. Clin Nutr. Apr, 29 (2): 154-9; Fielding et al. (2011) Sarcopenia: An Undiagnosed Condition in Older Adults. Current Consensus Definition: Prevalence, Etiology, and Consequences. International Working Group on Sarcopenia. J Am Med Dir Assoc, 12: 249-256; and Studenski et al. (2014) The FNIH Sarcopenia Project: Rationale, study description, conference recommendations and final estimates. J Gerontol A Biol Sci Med Sci 69 (5): 547-558).

Frailty is a geriatric condition characterized by an increased vulnerability to external stressors. It is strongly linked to adverse outcomes, including mortality, nursing home admission, and falls. In some embodiments, the muscle condition is a condition associated with one or more characteristic measures of frailty. In some embodiments, the subject is classified as frail. In some embodiments, the subject is classified as pre-frail, and is at a high risk or progression to being frail. Frailty can be diagnosed and/or characterized according to various indices of frailty that are composite measures of age-related changes indices of frailty, such as methods based on the Fried's frailty scale (see e.g., Fried, et al., Frailty in older adults: evidence for a phenotype. J Gerontol A Biol Sci Med Sci. 2001, 56: M146-M156) and/or the Mitnitski's Frailty Index (see e.g., Mitnitski et al., Frailty, fitness and late-life mortality in relation to chronological and biological age. BMC Geriatr. 2002, 2: 1-10).

In some embodiments, the muscle condition is muscle atrophy. Muscle atrophy refers to any wasting or loss of muscle tissue resulting from lack of use. Muscle atrophy can lead to muscle weakness and cause disability. In some embodiments, the muscle condition is immobilization-associated muscle weakness, which refers to any wasting or loss of muscle tissue resulting from immobilization, e.g., for medical reasons.

In some embodiments, the muscle condition is muscle weakness, also referred to as muscle fatigue, which refers to a condition characterized by the subject's inability to exert force with skeletal muscles. Muscle weakness often follows muscle atrophy.

Muscle atrophy can be measured using various endpoints, such as skeletal muscle protein fractional synthetic rate (FSR) in a liquid biopsy. Other measurements of muscle atrophy include diaphragm thickness, echo-density (e.g. of vastus lateralis), muscle circumference (of muscles such as the thigh/vastus lateralis), muscle cross-sectional area, and the like. Detection of muscle circumference can be measured using ultrasound. Ultrasound can be used to assess muscle atrophy, diaphragm dysfunction, predict extubating success or failure, quantify respiratory effort, and detect atrophy in, for example, mechanically ventilated subjects or subjects on bedrest.

In some embodiments, the muscle condition is a skeletal muscle condition. In some embodiments, the muscle condition is not a cardiovascular condition. In some embodiments, the subject is not suffering from, or identified as having, a cardiovascular disease or condition. In some embodiments, the subject is not suffering from, or at risk of, a heart failure.

In some embodiments the age-related muscle condition is associated with the loss-of-function, decrease in the ability to regenerate, or heal after injury of skeletal muscle. In some embodiments the age-related muscle condition is associated with the loss-of-function of muscle stem cells.

In some embodiments, the muscle condition is due to insulin insensitivity associated with muscle atrophy. Type 2 diabetes mellitus can be associated with an accelerated muscle loss during aging, decreased muscle function, and increased disability.

### 5.2.1. Patient Age

In some embodiments of the method of treating a subject for a muscle condition, the subject has, or is suspected of having, an age-related muscle condition.

In some embodiments, the subject is human. The subject can be a human patient suffering from, or a risk of, an age-related muscle condition. In some embodiments, the patient is at least 40-years-old. In some embodiments, the patient is at least 50-years-old. In some embodiments, the patient is at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 40-50 years old, 50-60 years old, 60-70 years old, 70-80 years old, or 80-90 years old.

### 5.2.2. Assessment of Patients

A subject can be identified as in need of treatment according to the methods of this disclosure, using a variety of different assessment methods.

A sarcopenia diagnosis can be determined or confirmed by the presence of low muscle quantity or quality. When low muscle strength or force, low muscle quantity/quality and low physical performance are all detected, sarcopenia is considered severe. In some embodiments, the patient has low muscle quantity or quality as compared to criteria representative of a healthy human subject, e.g., a subject of the same age or younger.

Low muscle mass can be assessed using appendicular lean body mass (ALBM). In some embodiments, low muscle mass is indicated by an ALBM adjusted for body mass index (BMI) of <0.789 kg for men or <0.512 kg for women, where ALBM can be measured by dual energy X-ray absorptiometry (DXA).

Low muscle mass can be assessed by the appendicular skeletal muscle index (ASMI). In some low muscle mass is indicated by an appendicular skeletal muscle index (ASMI) of less than 7.26 kg/m² for men, or less than 5.5 kg/m² for women, said ASMI being defined as appendicular skeletal muscle mass divided by the square of height, said ASMI being measured by dual energy X-ray absorptiometry (DXA).

Low muscle strength can include low grip strength, and be determined using a handgrip strength test. In some embodiments, low grip strength is assessed by measuring the amount of static force that the hand can squeeze around a handgrip dynamometer, e.g., as indicated by a value of less than 30 kg, such as less than 26 kg for men, or less than 20 kg for women, such as less than 16 kg, in the handgrip strength test.

In some embodiments, the human subject has, or is identified as having, low muscle strength. In some embodiments, the human subject has, or is identified as having, low muscle force.

In some embodiments, the human subject has, or is identified as having, low lower limb muscle mass. In some embodiments, the human subject has, or is identified as having, low upper limb muscle mass.

In some embodiments, the human subject has, or is identified as having, low muscle volume. In some embodiments, the muscle volume is skeletal muscle volume. In some embodiments, the muscle is a skeletal muscle. In some embodiments, the skeletal muscle is a diaphragm. In some embodiments, the muscle is diaphragm, tibialis anterior, tibialis posterior, gastrocnemius, sartorius, vastus intermedius, vastus laterals, vastus medialis, soleus, or extensor digitorum longus. In some embodiments, the muscle is diaphragm, tibialis anterior, tibialis posterior, sartorius, soleus, or extensor digitorum longus. In some embodiments, the muscle is diaphragm muscle.

In some embodiments, the muscle volume is the muscle volume of one or more upper limb muscles selected from the group consisting of: shoulder abductors, shoulder adductors, elbow flexors, elbow extensors, wrist flexors, and wrist extensors.

In some embodiments, muscle mass is assessed after the dosing. In some embodiments, muscle mass is assessed at least one day after dosing. In some embodiments, the muscle mass is assessed at least one week after dosing. In some embodiments, the muscle mass is assessed at least one month after dosing.

In some embodiments, the muscle condition is a skeletal muscle condition. In some embodiments, the skeletal muscle expresses the apelin receptor and administration of the apelin receptor modulator activates the apelin/APJ system (APLNR gene) in the muscle tissue of the subject. The muscle of interest expresses the apelin receptor, and in some embodiments, the level of expression of the apelin receptor can be assessed or determined in a muscle tissue of the subject prior to and/or after treatment. In some embodiments, the subject has, or is identified as having, a low circulating level of apelin. Apelin circulating levels can be assessed in a biological sample obtained from the subject, e.g., using a quantitative assay (e.g., ELISA assay, or LC/MS) for determining the amount of an apelin peptide in a sample.

In some embodiments, the muscle condition is a diaphragmatic muscle condition. In some embodiments, the diaphragmatic muscle condition is diaphragm atrophy. In some embodiments, the diaphragmatic muscle condition is diaphragm dysfunction. Dysfunction of the diaphragm ranges from a partial loss of the ability to generate pressure (weakness) to a complete loss of diaphragmatic function (paralysis). Patients with bilateral diaphragmatic paralysis or severe diaphragmatic weakness are likely to have dyspnea or recurrent respiratory failure. They can have considerable dyspnea at rest, when supine, with exertion, or when immersed in water above their waist. Further, patients with bilateral diaphragmatic paralysis are at an increased risk for sleep fragmentation and hypoventilation during sleep.

In some embodiments of the methods of this disclosure, the subject is human and has, or is identified as having, one or more of diabetes mellitus, insulin insensitivity, cardiovascular disease, and neurologic disease.

In some embodiments, the subject is human and has, or is identified to have diaphragm atrophy. In certain embodiments, the subject is human is undergoing mechanical ventilation (e.g. is mechanically ventilated at time of diagnosis). In certain embodiments, the subject is human and has, or is identified to have diaphragm atrophy caused by mechanical ventilation. In some embodiments, the subject is human and is on a ventilator (e.g. mechanical ventilatory).

In some embodiments of the methods of this disclosure, the subject is human and has, or is identified as having, hypoxic respiratory failure. Hypoxic respiratory failure can be measured by stratifying diaphragm thickness.

Muscle atrophy can be measured using various endpoints, such as skeletal muscle protein fractional synthetic rate (FSR) in a liquid biopsy. Other measurements of muscle atrophy include diaphragm thickness, echo-density (e.g. of vastus lateralis), muscle circumference (of muscles such as the thigh/vastus lateralis), muscle cross-sectional area, and the like. Detection of muscle circumference can be measured using ultrasound. Ultrasound can be used to assess diaphragm dysfunction, predict extubating success or failure, quantify respiratory effort, and detect atrophy in, for example, mechanically ventilated subjects.

Diaphragm atrophy can be measured by a change in diaphragm thickness. For example, diaphragmatic thickness can be measured in subjects that are mechanically ventilated before ventilation, at the time of ventilation, after a number of days on a ventilator, after treatment, and the like (see e.g., Schepens et al., (2015) Crit Care; 19: 422). In some embodiments, the human subject has, or is identified as having reduced diaphragm thickness as compared to a human subject that is not mechanically ventilated, or as compared to a baseline value for the subject prior to mechanical ventilation.

### 5.3. Methods of Maintaining Muscle Mass or Muscle Strength

Aspects of this disclosure include a method for maintaining and/or increasing muscle mass and/or muscle strength in an elderly subject. In various embodiments, an apelin receptor modulator (e.g., as described herein) is administered to the elderly subject to maintain or increase muscle mass and/or muscle strength in skeletal muscle of the subject. In some embodiments, the apelin receptor modulator is an apelin receptor agonist.

In some embodiments, the elderly subject is human and at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 60-70 years old, 70-80 years old, or 80-90 years old.

The muscle mass and/or muscle strength of a subject can be monitored during treatment and compared to a baseline assessment performed prior to dosing with the apelin receptor modulator. In some embodiments, the apelin receptor modulator is an apelin receptor agonist. In some embodiments, the muscle mass or muscle strength of a subject is at least maintained at baseline levels during treatment. In some embodiments, the subject is one who has suffered from declining muscle mass and/or muscle strength over time, and administration of the apelin receptor modulator according to methods of this disclosure reverses and/or ameliorates the decline. In some embodiments, the apelin receptor modulator is an apelin receptor agonist.

Low muscle mass can be assessed using appendicular lean body mass (ALBM). In some embodiments, low muscle mass is indicated by an ALBM adjusted for body mass index (BMI) of <0.789 kg for men or <0.512 kg for women, where ALBM can be measured by dual energy X-ray absorptiometry (DXA).

Low muscle mass can be assessed by the appendicular skeletal muscle index (ASMI). In some low muscle mass is indicated by an appendicular skeletal muscle index (ASMI) of less than 7.26 kg/m² for men, or less than 5.5 kg/m² for women, said ASMI being defined as appendicular skeletal muscle mass divided by the square of height, said ASMI being measured by dual energy X-ray absorptiometry (DXA).

Low muscle strength can be determined using a handgrip strength test. In some embodiments, low muscle strength is indicated by a value of less than 30 kg, such as less than 26 kg for men, or less than 20 kg for women, such as less than 16 kg, in the handgrip strength test.

In some embodiments, muscle mass is assessed before and after the dosing of the apelin receptor agonist. In some embodiments, the muscle mass is assessed at least one day after dosing. In some embodiments, the muscle mass is assessed at least one week after dosing. In some embodiments, the muscle mass is assessed at least one month after dosing.

In some embodiments, muscle strength is assessed before and after the dosing of the apelin receptor agonist. In some embodiments, the muscle strength is assessed at least one day after dosing. In some embodiments, the muscle strength is assessed at least one week after dosing. In some embodiments, the muscle strength is assessed at least one month after dosing.

In some embodiments, the subject has, or is identified as having, a low circulating level of apelin. Apelin circulating levels can be assessed in a biological sample obtained from the subject.

### 5.4. Apelin Receptor Modulators

Apelin is the endogenous ligand for the apelin receptor (also referred to as APJ, or APLNR). The apelin receptor is a member of the rhodopsin-like G protein-coupled receptor (GPCR) family. The apelin/APJ system is distributed in diverse periphery organ tissues and can play various roles in the physiology and pathophysiology of many organs. The apelin/APJ system participates in various cell activities such as proliferation, migration, apoptosis or inflammation. An apelin receptor modulators can activate the APJ system directly or indirectly, competitively, or non-competitively.

As further described below, in some embodiments of the methods of this disclosure, the apelin receptor modulator (e.g., apelin receptor agonist) is a compound described in U.S. Pat. Nos. 9,573,936 or 9,868,721, the disclosures of which are herein incorporated by reference in their entirety.

As known by those skilled in the art, certain compounds of this disclosure may exist in one or more tautomeric forms. Because one chemical structure may only be used to represent one tautomeric form, it will be understood that for convenience, referral to a compound of a given structural formula includes tautomers of the structure represented by the structural formula.

In some embodiments, the apelin receptor modulator is a compound of formula (I) or (II):

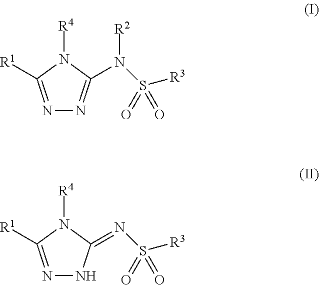

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)—OH, —O—(C₁-C₆ alkyl)-O—(C₁-C₆ alkyl), —O—(C₁-C₆ haloalkyl)—OH, —O—(C₁-C₆ haloalkyl)-O—(C₁-C₆ alkyl), —O—(C₁-C₆ perhaloalkyl)—OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂-(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}—CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

In some embodiments, the apelin receptor modulator is a compound of formula (I) or (II):

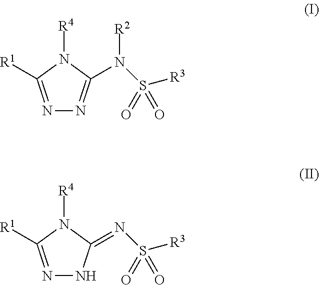

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)—OH, —O—(C₁-C₆ alkyl)-O—(C₁-C₆ alkyl), —O—(C₁-C₆ haloalkyl)—OH, —O—(C₁-C₆ haloalkyl)-O—C₁-C₆ alkyl), —O—(C₁-C₆ perhaloalkyl)—OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —C(═O) O—(C₁-C₆ alkyl), C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, or S;
R² is selected from —H, or C₁-C₄ alkyl or is absent in the compounds of Formula II;
R³ is a group of formula —(CR^{3b}R^{3c})—(CR^{3f}R^{3g})-Q;
R^{3d} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), or —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), or —N(C₁-C₆ alkyl)₂;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, or a heteroaryl group, and the Q heterocyclyl group may be substituted with 1 oxo substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, or S, or a monocyclic or bicyclic heterocyclyl group with 5 to 10 ring members containing 1, 2, 3, or 4 heteroatoms independently selected from N, O, or S, wherein the C₆-C₁₀ aryl group, the heteroaryl group, or the heterocyclyl group are unsubstituted or are substituted with 1, 2, or 3 R^{4a} substituents; and
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(O)NH(C₁-C₆ alkyl), or —C(═O)N(C₁-C₆ alkyl)₂, and the heterocyclyl R⁴ group may be further substituted with 1 oxo substituent.

As noted above, apelin receptor agonist compounds of this disclosure may exist in multiple tautomeric forms. This is particularly true in compounds of Formula I where R² is H. These forms are illustrated below as Tautomer A and Tautomer B:

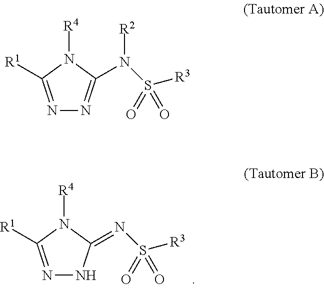

Apelin receptor agonist compounds of this disclosure are depicted structurally and generally named as compounds in the “Tautomer A” form. However, it is specifically contemplated and known that the compounds exist in “Tautomer B” form and thus compounds in “Tautomer B” form are expressly considered to be part of this disclosure. For this reason, the claims refer to compounds of Formula I and Formula II. Depending on the compound, some compounds may exist primarily in one form more than another. Also, depending on the compound and the energy required to convert one tautomer to the other, some compounds may exist as mixtures at room temperature whereas others may be isolated in one tautomeric form or the other.

In some embodiments of formula (I) and (II), R¹ is an unsubstituted pyridyl or is a pyridyl substituted with 1 or 2 R^{1a} substituents.

In some embodiments of formula (I) and (II), R^{1a} in each instance is independently selected from —CH₃, —CH₂CH₃, —F, —Cl, —Br, —CN, —CF₃, —CH═CH₂, —C(═O)NH₂, —C(═O)NH(CH₃),—C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

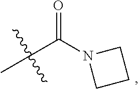 [SMILES: *C(=O)N1CCC1]

wherein the symbol [symbol] , when drawn across a bond, indicates the point of attachment to the rest of the molecule.

In some embodiments of formula (I) and (II), R¹ is selected from

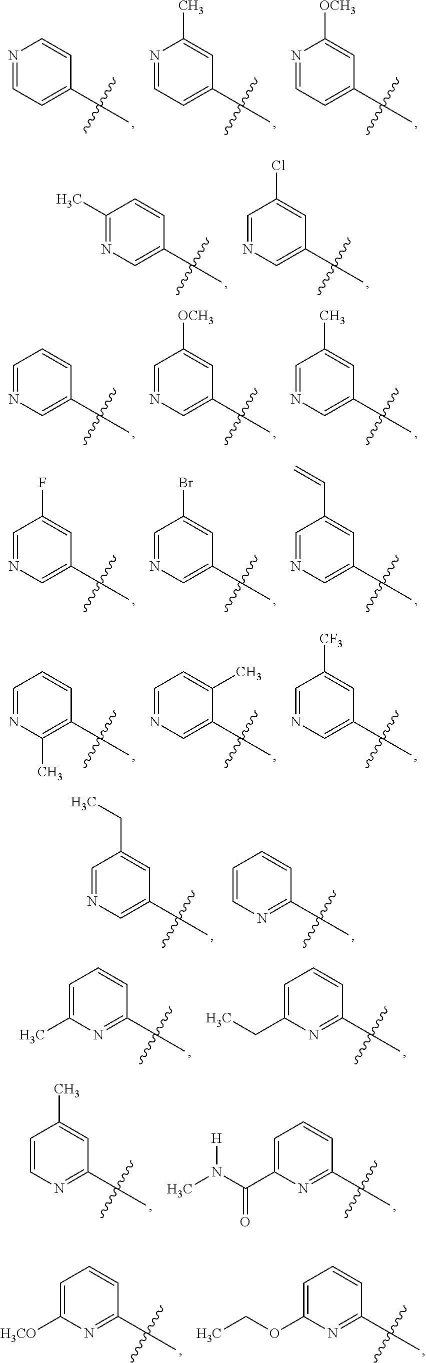

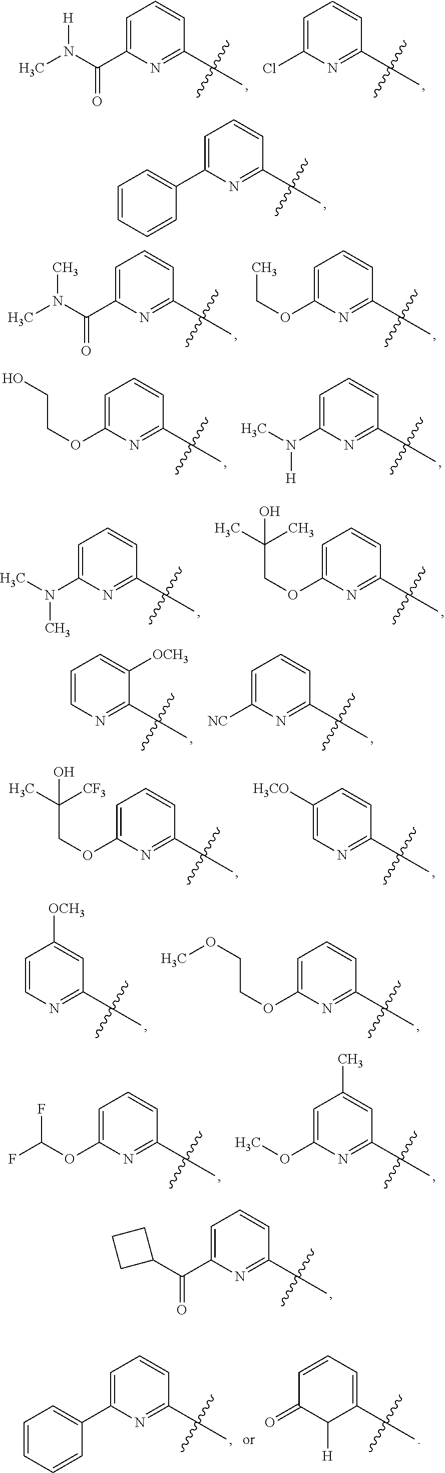

wherein the symbol [symbol] , when drawn across a bond, indicates the point of attachment to the rest of the molecule.

In some embodiments of formula (I) and (II), R¹ is selected from

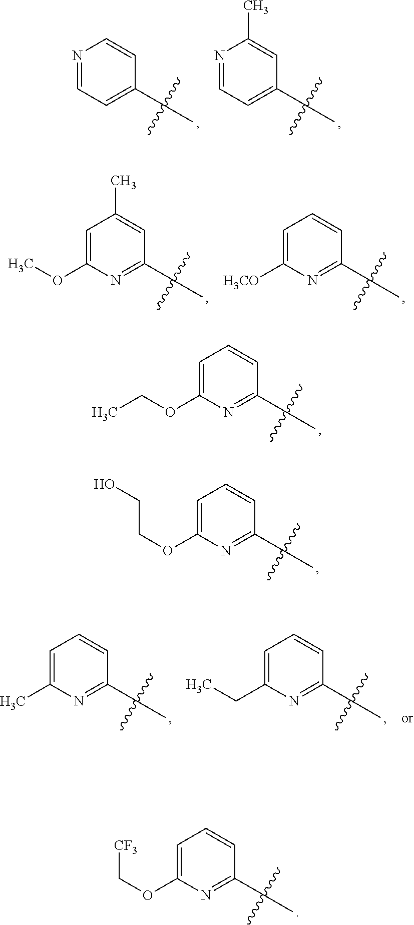

wherein the symbol [symbol] ,when drawn across a bond, indicates the point of attachment to the rest of the molecule.

In some embodiments of formula (I) and (II), R² is —H.

In some embodiments of formula (I) and (II), R⁴ is a phenyl, pyridyl, pyrimidinyl, isoxazolyl, indolyl, naphthyl, or pyridinyl any of which may be unsubstituted or substituted with 1, 2, or 3 R^{4a} substituents. In some embodiments of formula (I) and (II), R⁴ is a phenyl substituted with 1 or 2 R^{4a} substituents. In some embodiments of formula (I) and (II), the 1 or 2 R^{4a} substituents are —O—(C₁-C₂ alkyl) groups.

In some embodiments of formula (I) and (II), R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

In some embodiments of formula (I) and (II), R⁴ is selected from:

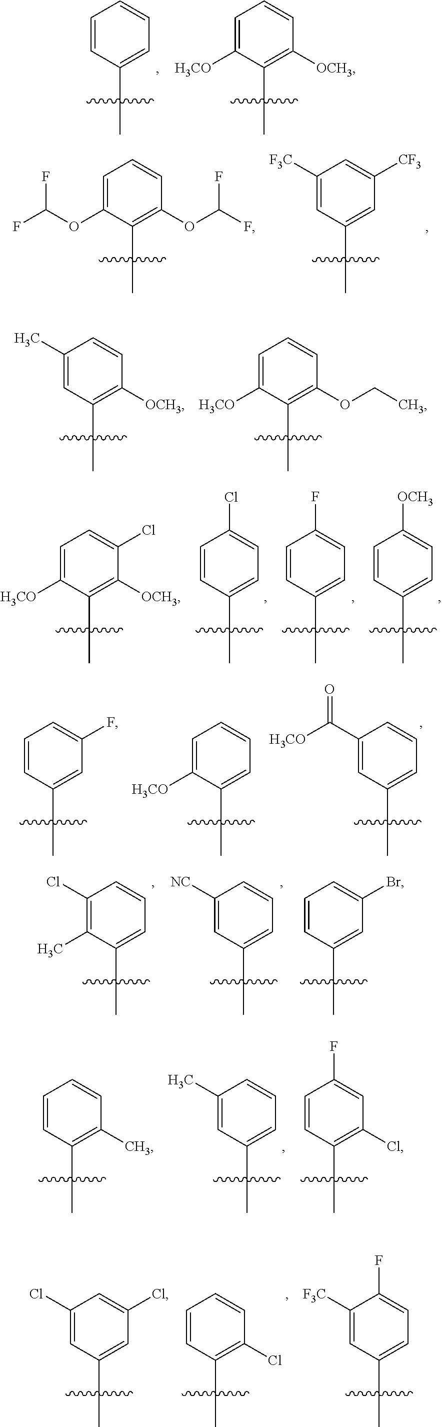

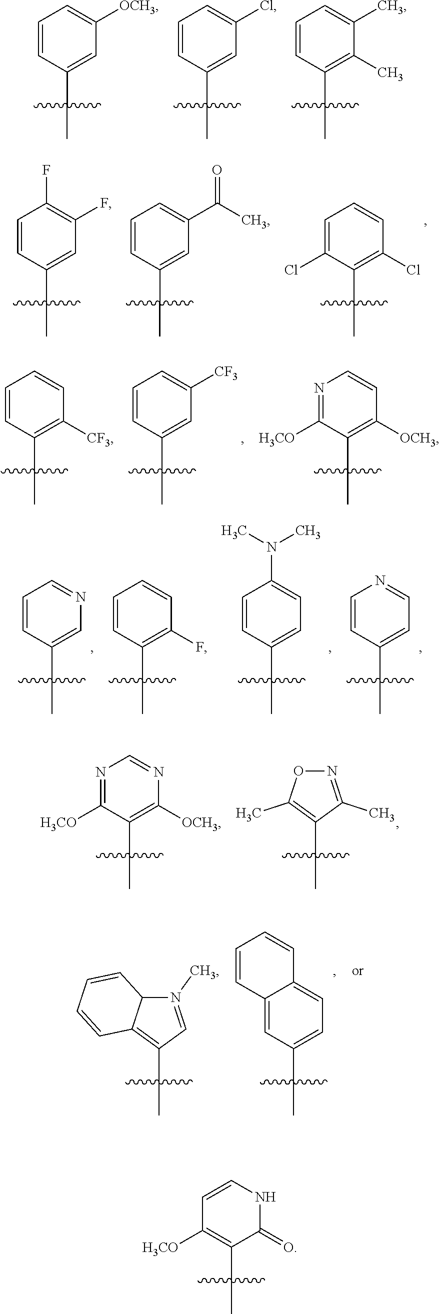

wherein the symbol [symbol] , when drawn across a bond, indicates the point of attachment to the rest of the molecule.

In some embodiments of formula (I) and (II), R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}—CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

In some embodiments of formula (I) and (II), Q is a monocyclic heteroaryl group with 5 or 6 ring members containing 1 or 2 heteroatoms selected from N, O, or S and Q is unsubstituted or is substituted with 1 or 2 R^{Q} substituents.

In some embodiments of formula (I) and (II), Q is selected from

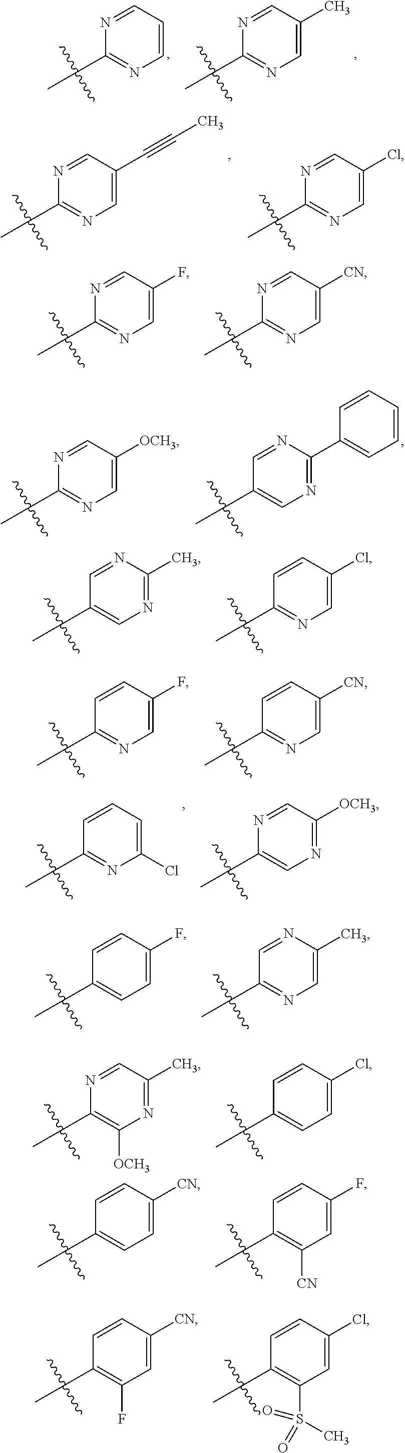

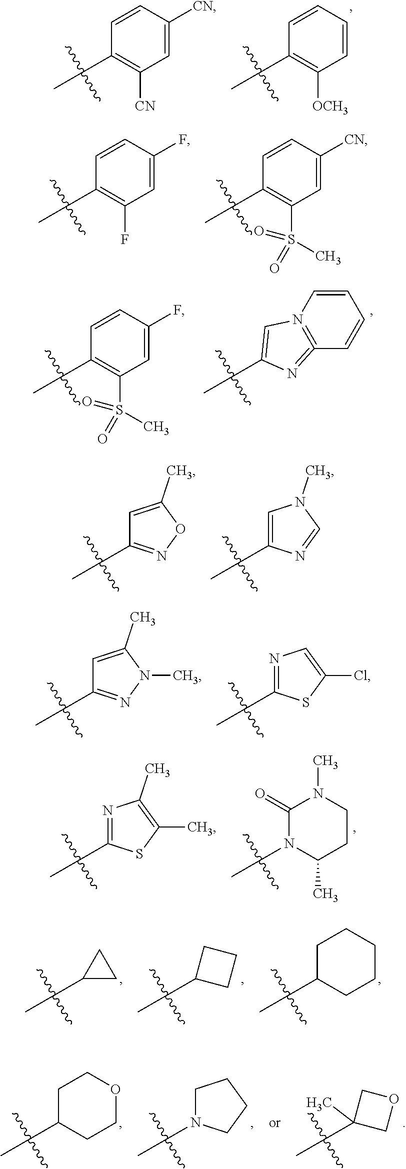

wherein the symbol [symbol] , when drawn across a bond, indicates the point of attachment to the rest of the molecule.

In some embodiments of formula (I) and (II), R³ is a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), R³ is a group of formula —(CR^{3d}R^{3e})—(CR^{3f}R^{3g})-Q.

In some embodiments of formula (I) and (II), R³ has the formula

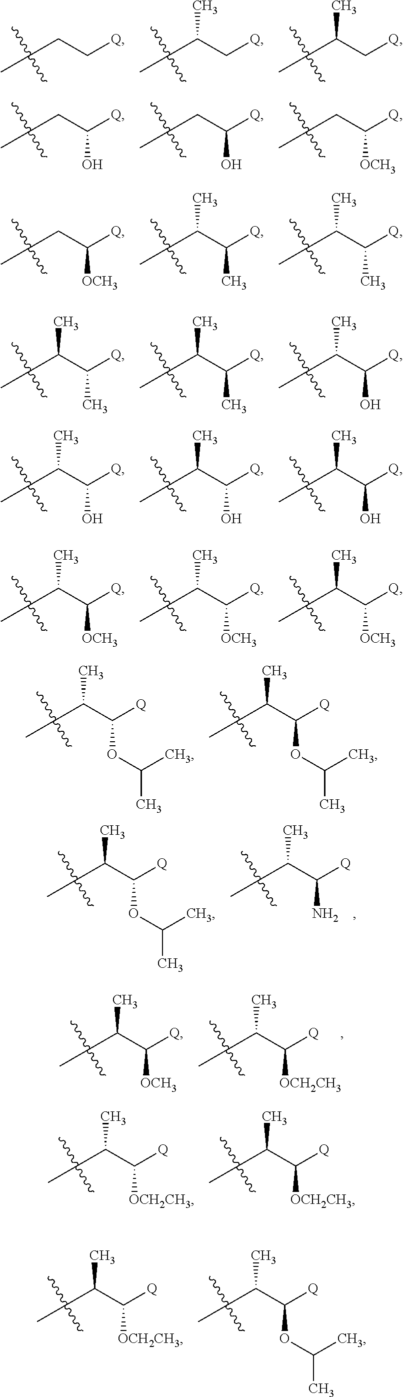

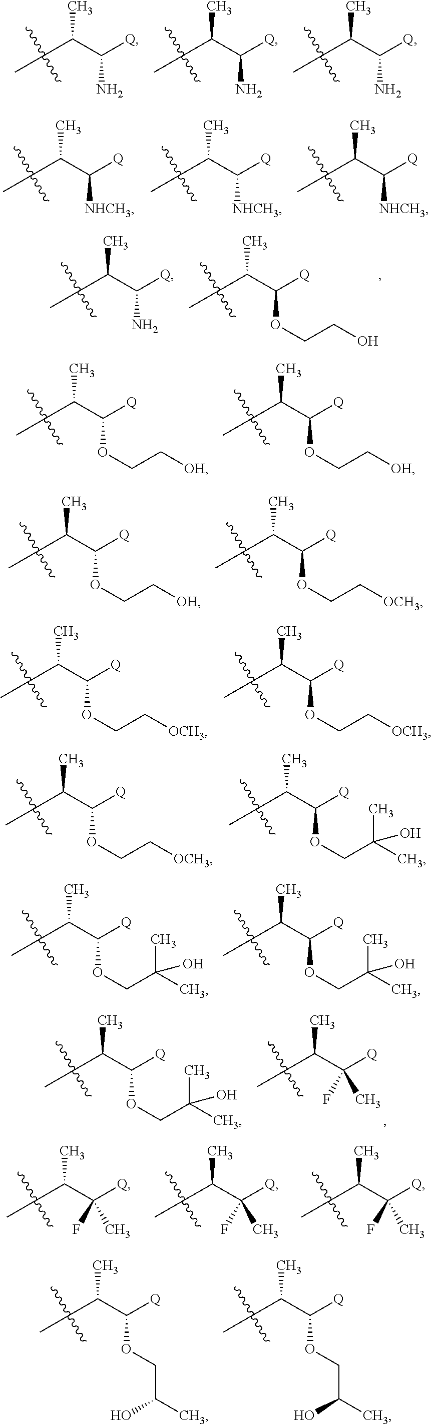

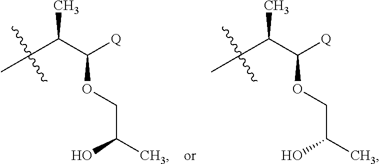

wherein the symbol [symbol] , when drawn across a bond, indicates the point of attachment to the rest of the molecule.

In some embodiments of formula (I) and (II), R³ has the formula

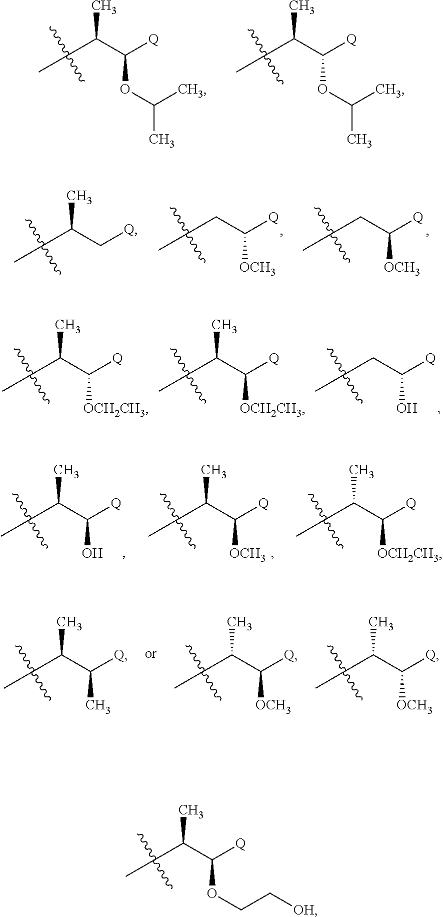

wherein the symbol [symbol] , when drawn across a bond, indicates the point of attachment to the rest of the molecule.

In particular embodiments of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide; (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide; (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide; (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide; (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide; (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide; (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide; (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide; (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide; (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide; (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide; (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide; (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide; (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide; (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide; (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide;

(1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide; (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide; (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide; (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide; (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide; (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide; (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide; (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide; (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide; or (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide or the pharmaceutically acceptable salt thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propane sulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2, 6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2, 6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5- methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is

(1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-difluorophenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide (BGE-105) or a pharmaceutically acceptable salt thereof.

In a particular embodiment of formula (I) and (II), the apelin receptor agonist is

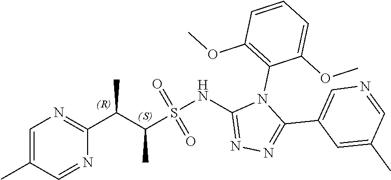

(BGE-105) or a pharmaceutically acceptable salt thereof.

U.S. Pat. Nos. 9,573,936, 9,868,721, 9,745,286, 9,656,997, 9,751,864, 9,656,998, 9,845,310, 10,058,550, 10,221,162, and 10,344,016, the disclosures of which are incorporated herein by reference in their entirety, describe apelin receptor agonists of formula (I) or (II), and methods of synthesizing such triazole agonists of the apelin receptor, including BGE-105. See e.g., Example 263.0 of U.S. Pat. No. 9,573,936.

If any variable occurs more than one time in a chemical formula, its definition on each occurrence is independent of its definition at every other occurrence. If the chemical structure and chemical name conflict, the chemical structure is determinative of the identity of the compound. The compounds of this disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Certain compounds of this disclosure may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R⁴ is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

Unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed.

Various compounds of this disclosure contain one or more chiral centers, and can exist as racemic mixtures of enantiomers, mixtures of diastereomers or enantiomerically or optically pure compounds. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents.

Compounds of the present disclosure include, but are not limited to, compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. The term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

The compounds of this disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

The term “pharmaceutically acceptable salt” refers to a salt that is acceptable for administration to a subject. Examples of pharmaceutically acceptable salts include, but are not limited to: mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glycine salt, lysine salt, arginine salt, ornithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt. The term “salt(s)” as used herein encompass hydrate salt(s).

Other examples of pharmaceutically salts include anions of the compounds of the present disclosure compounded with a suitable cation. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Furthermore, if the compounds of the present invention or salts thereof form hydrates or solvates, these are also included in the scope of the compounds of the present invention or salts thereof.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

### 5.4.1. Pharmaceutical Composition

The apelin receptor agonist compounds used in the methods described herein can be formulated in any appropriate pharmaceutical composition for administration by any suitable route of administration. The pharmaceutical compositions can include the compound or the pharmaceutically acceptable salt thereof, the tautomer thereof, the pharmaceutically acceptable salt of the tautomer, the stereoisomer of any of the foregoing, or the mixture thereof according to any one of the embodiments described herein and at least one pharmaceutically acceptable excipient, carrier or diluent. In some such embodiments, the compound or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of a muscle condition (e.g., as described herein), for activating the APJ receptor.

Suitable routes of administration include, but are not limited to, oral, topical, and intravenous routes of administration. Suitable routes also include pulmonary administration, including by oral inhalation. The most suitable route may depend upon the condition and disorder of the recipient. The formulations may conveniently be presented in unit dosage form and may be prepared by any of the methods known in the art of pharmacy.

In some embodiments, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a tablet where the effective amount of the active ingredient ranges from 5 mg to 60 mg, from 6 mg to 58 mg, from 10 mg to 40 mg, from 15 mg to 30 mg, from 16 mg to 25 mg, or from 17 mg to 20 mg. In some such compositions, the amount of active ingredient is 17 mg.

All methods include the step of bringing into association an apelin agonist, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein is parenteral administration. In certain embodiments, the route of administration for use in the methods described herein is intravenous administration (e.g., intravenous infusion). In certain embodiments, the route of administration for use in the methods described herein is oral administration. In certain embodiments, the route of administration for use in the methods described herein is constant intravenous infusion.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain antioxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. Any suitable pharmaceutical excipient may be used, and one of ordinary skill in the art is capable of selecting suitable pharmaceutical excipients. Pharmaceutical excipients include, for example, those described in the Handbook of Pharmaceutical Excipients, 8th Revised Ed. (2017).

### 5.4.2. Dosage Regimens

In various embodiments, the apelin receptor agonist (e.g., as described herein) is administered at a dose sufficient to treat an age-related muscle condition (e.g., as described herein).

In various embodiments, the apelin receptor agonist (e.g., as described herein) is administered in a method for maintaining and/or increasing muscle mass and/or muscle strength in an elderly subject. In some embodiments, the elderly subject is human and at least 50 years old, at least 55 years old, at least 60-years-old, or at least 65 years old.

In various embodiments, the dose of the apelin receptor agonist is at least 0.01 mg/kg, such as at least 0.5 mg/kg, or at least 1 mg/kg. In certain embodiments, the dose is 25 mg/kg to 1,000 mg/kg per day.

In some embodiments, the apelin receptor agonist is administered in a dose that is independent of patient weight or surface area (flat dose).

In various embodiments, the dose is 1-5000 mg. In various embodiments, the dose is 25-2000 mg. In some embodiments, the dose is at least 60 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 420 mg, at least 440 mg, at least 460 mg, at least 480 mg, at least 500 mg, at least 520 mg, at least 550 mg, at least 580 mg, at least 600 mg, at least 650 mg, at least 700 mg, at least 750 mg, at least 800 mg, at least 850 mg, at least 900 mg, at least 950 mg, at least 1000 mg, at least 1100 mg, at least 1200 mg, at least 1300 mg, at least 1400 mg, or at least 100 mg. In various embodiments, the dose is 25-2000 mg. In some embodiments, the dose is at least 200 mg.

The apelin receptor agonist can be administered in a single dose or in multiple doses.

In some embodiments, the dose is administered daily.

In some embodiments, the dose is administered as a plurality of equally or unequally divided sub-doses.

In certain embodiments, the dose is administered continuously (e.g., IV infusion) for a period of time. In certain embodiments, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours). In certain embodiments, following the dose, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a dose and a 24 hour or 48-hour washout period, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a first dose and a 24 hour or 48-hour washout period, the dose is administered as an intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours), followed by a second dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours).

In some embodiments, the apelin receptor agonist is administered orally, intravenously, intranasally, or intramuscularly. In some embodiments, the apelin receptor agonist is administered orally.

In some embodiments, the apelin receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the apelin receptor agonist is administered continuously for at least 10 minutes, at least 20 minutes, at least 30 minutes, at least 40 minutes, at least 50 minutes, at least 1 hour, at least 2 hours, at least 3 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 48 hours, at least 72 hours, at least 100 hours, at least 110 hours, at least 115 hours, at least 120 hours, or at least 125 hours.

### 5.4.3. Dosage Form

In some embodiments, an apelin receptor modulator or salt thereof is administered in a suspension. In other embodiments, an apelin receptor modulator or salt thereof is administered in a solution. In some embodiments, an apelin receptor modulator or salt thereof is administered in a solid dosage form. In particular embodiments, the solid dosage form is a capsule. In particular embodiments, the solid dosage form is a tablet. In specific embodiments, an apelin receptor modulator is in a crystalline or amorphous form. In particular embodiments, an apelin receptor modulator is in amorphous form. In some embodiments, the apelin receptor modulator is an apelin receptor agonist.

In one aspect of the methods, the apelin receptor modulator, or the pharmaceutical composition including same, is administered intravenously, topically, orally, by inhalation, by infusion, by injection, intraperitoneally, intramuscularly, subcutaneously, intra-aurally, by intra-articular administration, by intra-mammary administration, by topical administration or by absorption through epithelial or mucocutaneous linings. In certain embodiments, the apelin receptor modulator, or the pharmaceutical composition including same, is administered via intravenous infusion.

### 5.5. Definitions

Unless defined otherwise, all technical and scientific terms used herein have the meaning commonly understood by a person skilled in the art to which this invention belongs.

The terms “individual,” “host,” and “subject” are used interchangeably, and refer to an animal to be treated, including but not limited to humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations. The term “patient” refers to a human subject.

The term “modulator” refers to a compound or composition that modulates the level of a target, or the activity or function of a target, which may be, but is not limited to, apelin receptor. In some embodiments, the modulator compound can agonize or activate the target, such as apelin receptor. An agonist or activator of a target can increase the level of activity or signaling associated with the target.

The terms “treating,” “treatment,” and grammatical variations thereof are used in the broadest sense understood in the clinical arts. Accordingly, the terms do not require cure or complete remission of disease, and the terms encompass obtaining any clinically desired pharmacologic and/or physiologic effect, including improvement in physiologic measures associated with “normal”, non-pathologic, aging. Unless otherwise specified, “treating” and “treatment” do not encompass prophylaxis.

The phrase “therapeutically effective amount” refers to the amount of a compound that, when administered to a mammal or other subject for treating a disease, condition, or disorder, is sufficient to effect treatment of the disease, condition, or disorder. The “therapeutically effective amount” may vary depending on the compound, the disease and its severity and the age, weight, etc., of the subject to be treated.

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remain operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that is generally safe, non-toxic and neither biologically nor otherwise undesirable, and include an excipient, diluent, carrier, and adjuvant that is acceptable for veterinary use as well as human pharmaceutical use. The phrase “pharmaceutically acceptable excipient” includes both one and more than one such excipient, diluent, carrier, and/or adjuvant.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an -OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group.

“Alkynyl” refers to an unsaturated branched or straight-chain hydrocarbon having at least one carbon-carbon triple bond derived by the removal of one hydrogen atom from a single carbon atom of a parent alkyne. Typical alkynyl groups include, but are not limited to, ethynyl; propynyl; butynyl, 2-pentynyl, 3-pentynyl, 2-hexynyl, 3-hexynyl and the like. In certain embodiments, an alkynyl group has 2 to 20 carbon atoms and in other embodiments, has 2 to 6 carbon atoms. An alkynyl group having 2 to 6 carbon atoms may be referred to as a —(C₂-C₆)alkynyl group.

“Alkoxy” refers to a radical -OR where R represents an alkyl group as defined herein. Representative examples include, but are not limited to, methoxy, ethoxy, propoxy, butoxy, cyclohexyloxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to the radical —C(O) or —C(═O) group.

“Carboxy” refers to the radical —C(O)OH.

“Cyano” refers to the radical —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₈)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

“Heterocyclyl” refers to a cyclic group that includes at least one saturated or unsaturated, but non-aromatic, cyclic ring. Heterocyclyl groups include at least one heteroatom as a ring member. Typical heteroatoms include O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring.

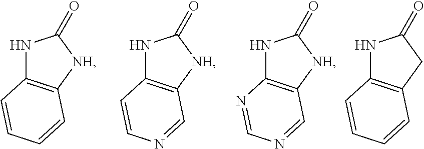

In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatoms are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Substituted heterocyclyl also includes ring systems substituted with one or more oxo (═O) or oxide (—O⁻) substituents, such as piperidinyl N-oxide, morpholinyl-N-oxide, 1-oxo-1-thiomorpholinyl, pyridinonyl, benzimidazolonyl, benzo[d]oxazol-2(3H)-only, 3,4-dihydroisoquinolin-1(2H)-only, indolin-only, 1H-imidazo[4,5-c]pyridin-2(3H)-only, 7H-purin-8(9H)-only, imidazolidin-2-only, 1H-imidazol-2(3H)-only, 1,1-dioxo-1-thiomorpholinyl, and the like.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2 In certain embodiments, the total number of S and O atoms in the aromatic heterocycle is not more than 1 Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 6. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature

### 6.1. Example 1: Bioinformatic Analyses Identify Relationships Between Apelin and All-Cause Mortality and Mobility Decline Events in Human Healthy Aging Cohorts

A survival predictor model was used to examine the relationship between serum levels of apelin and future risk of all-cause mortality in human healthy aging cohorts, using unpublished clinical outcome data and proteomics data generated on archived samples, based on survival modeling. Additionally, the relationship between apelin levels and mobility decline events (e.g., a decrease in walking, stair-climbing, or transferring activities indicated by self-reported difficulty of these activities) was examined. A Cox proportional hazards model was used, with a hazard ratio and associated p-value generated for apelin.

As shown in FIG. 2A, a Kaplan-Meier curve of survival probability was generated for humans in the top 20% (blue) versus bottom 20% (red) of apelin protein levels. In humans, we have discovered that higher circulating levels of apelin are associated with decreased all-cause mortality (p=0.0002). FIG. 2B shows a similar model, where we discovered that higher circulating levels of apelin are associated with increased mobility (p=0.0082). The hazard ratio for apelin (0.88 in FIG. 2A and 0.89 in FIG. 2B) was generated using a Cox proportional hazards model. p-values in FIGS. 2A and 2B were calculated for these hazard ratios, based on testing the null hypothesis that the hazard ratio in each case equals 1.

Next, the protein levels were subjected to a rank-based inverse normalization and calculated pairwise Spearman correlations coefficient between normalized levels of all 4,575 proteins. Among the 590 proteins that were significantly correlated (Benjamini-Hochberg FDR<0.05) with apelin (referred to as the apelin protein module; FIG. 2C), a hypergeometric test revealed significant enrichment of proteins associated with all-cause mortality (p=1.04E−10).

A multivariate Cox regression model was then used to test for association between the first principal component (PC1) of the apelin protein module and death rate after adjusting for age, pack-years smoked, and monthly alcohol consumption. The contribution of PC1 to the relative death rate in this model (i.e., the termplot), with the median PC1 value used as a reference, ranged from 1.43 to 0.77 (FIG. 2D).

FIG. 2C, shows the serum abundance of the apelin protein module (highlighted by the green oval) in the HHS cohort. Each node represents a protein, and the edges between the nodes represent significant correlations. FIG. 2D shows the first principal component of the apelin protein module and death rate. The relative death rate (log; y-axis) was derived from the multivariate Cox regression model for the PC1 after adjusting for age, smoking pack years, and alcohol status. The reference used was the median value of PC1.

### 6.2. Example 2: BGE-105 Improves Activity Levels in Old Mice (Frailty Study)

Based on the discovery of the association of baseline apelin and apelin receptor protein levels with future aging outcomes in otherwise healthy, aged, humans as described in Example 1, an agonist of apelin receptors was administered to elderly mice to assess the effects of the agonist on voluntary physical activity as compared to age-matched controls.

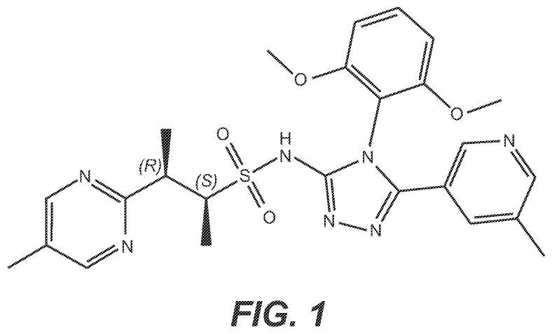

BGE-105 has the structure shown below (FIG. 1):

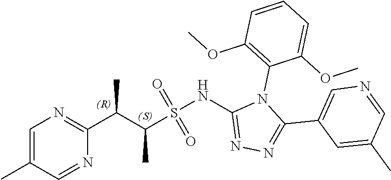

BGE-105 is known to activate the apelin receptor and it induces a cardiovascular response in rats (Ason et al., JCI Insight. 5 (8): 1-16 (2020)). Clinical trials were also done with BGE-105 to study the safety, tolerability, and pharmacokinetics in healthy subjects and those with suffering impaired renal function (NCT03318809) or heart failure (NCT03276728).

In the current study, aged (24-month-old) mice were treated with BGE-105 daily (in water ad libitum) for 2 months. The animals were housed with access to voluntary running wheels that wirelessly transmit running data to a computer for analysis. Voluntary running wheel activity levels were measured daily, and body weights were measured every 2 weeks. The effects of BGE-105 on the prevention of frailty in mice were examined.

The formal test involve d calculating a Spearman correlation coefficient between these daily differences and the day number (e.g., days 1, 2, 3, etc. of the experiment) and testing the null hypothesis that this correlation coefficient equals 0.

The first day of the study (Study Day 1) started with animal acclimation, followed by the BGE-105 treatment start date on Study Day 19 (Phase Day 1). The study concluded on Study Day 83. Activity wheel monitoring started on Study Day 1 and ended on Study Day 83 (Phase Day 64). The data was analyzed at the end of the study. The total duration of activity monitoring after BGE-105 treatment initiation was 64 days. For the frailty portion of the study, mice were assessed using an activity monitoring wheel which was monitored passively with a computer monitoring system.

As shown in Table 1, the study included 23-24-month-old mice from strain C57BL/6. It is known that mice ranging from 18-24 months of age correlate with humans ranging from 56-69 years of age, with mice older than 24 months correlating with humans beyond 69 years old (Flurkey, Currer, and Harrison, 2007. “The mouse in biomedical research” in James G. Fox (ed.), American College of Laboratory Animal Medicine series, Elsevier, AP: Amsterdam; Boston). This age range meets the definition of “old,” defined as the presence of senescent changes in biomarkers in animals.

TABLE 1
Test Articles

| | | | | Number of | Dose Regimen | Dose | Dose |
|---|---|---|---|---|---|---|---|
| Cohort | Strain | Category | Age | Animals | and Route | Volume | Concentration |
| BGE-105 | C57bl/6 | E | 23-24 | 20 ± 0 | In Drinking | ad libitum | 275 ug/mL |
| | | | Months | | 8.5 pH Water | | |
| Vehicle | C57bl/6 | E | 23-24 | 20 ± 0 | In Drinking | ad libitum | 0 ug/mL |
| | | | Months | | 8.5 pH Water | | |

Mice were treated with BGE-105 at a dose concentration of 275 ug/mL. BGE-105 was dissolved in deionized water at 275 ug/mL. BGE-105 was administered in drinking water consumed ad libitum. The compound is mildly acidic when dissolved, resulting in a pH 4.5 solution. The deionized water was adjusted to pH 8.5 by adding 1N NaOH. The vehicle control group consumed water (ad libitum) of the same pH without drug.

The study parameters for Groups 1-2 are provided in Table 2. The study parameters for mice in Groups 1-2 included animal acclimation, animal welfare, such as checking the weight of the animal, clinical examination, administering the treatment, activity monitoring, and blood collection, on the particular Study Days and/or Phase Days.

TABLE 2
Procedure Schedule

| | | |
|---|---|---|
| Phase Day (PD)/ | Experimental Arm/ | |
| Study Day (SD) | Group | Description |

Acclimation

| | | |
|---|---|---|
| SD 1-7 | Acclimation-1: | Weigh mice at beginning and |
| | All Groups | end of week |
| | | Weigh control water |
| | | Clinical Examination |
| | | Single house in Mouse Cages |
| | | with Activity Monitoring Wheel |
| | | Exclude Nestlets, Innodome, |
| | | Innowheel |
| SD 8-19 | Acclimation-2: | Weigh mice |
| | All Groups | Clinical Examination |
| | | Insert a bottle of control water |
| | | with a pH of 8.5 |
| | | Weigh water bottle once every |
| | | 48 hours |
| | | Activity Monitoring Wheel |
| | | Begin monitoring 24-hour |
| | | activity monitoring wheel |
| | | Exclude Nestlets, Innodome, |
| | | Innowheel |
| SD 19 | Active: | Grid Hang Test |
| | All Groups | 1 trial (baseline values before |
| | | treatment begins) |

Treatment

| | | |
|---|---|---|
| PD 1-7/SD 20-26 | Active: | Randomize animals with |
| | All Groups | consideration of behavior tests, |
| | | Smart Wheel data and Body |
| | | Weights. |
| | | Weigh mice once every week for |
| | | the first 2 weeks, then on an as |
| | | needed basis |
| | | Clinical Examination |
| | | Begin treating the experimental |
| | | group with BGE-105 treated water |
| | | for the duration of the study at a pH |
| | | of 8.5. |
| | | Control group continues to drink pH |
| | | matched control water. |
| | | weigh water bottle once every 48 |
| | | hours |
| | | Check the pH of BGE-105 and |
| | | vehicle when prepared fresh and |
| | | after 2 weeks at RT before replacing |
| | | with freshly prepared bottles |
| PD 8-65/SD 26-83 | Active: | Weigh mice once every week |
| | All Groups | Clinical Examination |
| | | Monitor the amount of food in each |
| | | dish once per day |
| | | Activity Monitoring Wheel: |
| | | Continue activity monitoring |
| | | Continue BGE-105 vs control |
| | | treatment as above |
| | | Weigh water bottle once every week |
| | | Monitor mice daily through cage for |
| | | clinical signs and to ensure wheel is |
| | | not blocked by debris |
| | | Minimize interaction with mice and |
| | | allow them to express their natural |
| | | activity |
| PD all/SD all | Active: | Change cages as necessary |
| | All Groups | |
| PD 65/SD 80 | Active: | Activity Monitoring Wheel: |
| | All Groups | End activity monitoring. |
| | | Grid Hang Test |
| | | 1 trial (final values) |

| | |
|---|---|
| Humane Endpoint: | If animal meets humane endpoint: |
| (criteria for euthanasia): | Notified study director and take note |
| 1. Moribund | of time and cage number and |
| a. Indicated by: BCS = 1 for 48 consecutive hours OR | euthanize animal as appropriate |
| 25% weight loss OR severely hunched posture with | |
| squinting eyes OR inability to self-right | |
| 2. Agonal breathing/cyanosis | |
| 3. Inability to eat or drink | |
| 4. Severe diarrhea | |
| 5. Severe bleeding from any orifice | |
| 6. Severe convulsions from which the animal does not | |
| recover (e.g. status epilepticus) | |
| 7. Self-induced trauma (e.g. autotomy) | |
| Animal Found Dead: | If an animal is found dead: |
| | Notified study director and |
| | take note of time and cage |
| | number |

### Activity Monitoring Wheel Test

The activity-monitoring wheel is a running disk that monitors rotations. The wheel is capable of monitoring voluntary wheel running 24 hours a day. Activity was monitored passively and wirelessly with a computer monitoring system. Running wheel activity levels were monitored daily. The wheel data was reported as the daily median rotations in each group (BGE-105 treated vs. controls). Mouse activity levels were measured as the number of wheel revolutions per day for each mouse and converted into a daily count of kilometers run using the diameter of the wheel. Within each experimental group, the daily median value for activity was calculated. For each experiment, a baseline period before experiment start was used to calculate median baseline activity levels for each mouse. These baselines were subtracted from future measurements for the same mouse. The resulting daily-corrected medians during the experiment were plotted for each day of the experiment and a smoothed curve was drawn using local regression (LOESS). The daily differences between the distances run in each group were calculated and tested for an increasing trend using Kendall's rank correlation tau.

### Study Results

As shown in FIG. 3A, BGE-105-treated mice were significantly more active than controls (Kendall rank correlation tau p-value=0.00228). This study was performed twice, yielding similar results in each replicate (FIG. 3D, Kendall rank correlation tau p-value=1.14e−04).

Activity levels decreased in both groups of elderly mice over the course of these experiments (FIGS. 3A and FIG. 3D), but decreased significantly less in the treated group, resulting in progressive divergence of the activity curves for the drug-treated and vehicle-treated group. In both experiments, by the end of the experimental period, BGE-105-treated mice ran on average at least 1 km/day more than mice treated with vehicle.

### Grid Hang Test

Four 20-gallon plastic buckets were used to suspend a three-by-three-foot metal grid suspended approximately three feet from the ground. The ground just below the grid was padded with soft material. The metal grid was placed on its side so that it was perpendicular to the buckets' surface. The mouse was placed on the grid and carefully lowered so that the mouse began to hang. Once the grid was completely parallel with the horizontal plane (i.e. the floor), the timer was started. The timer was stopped when the mouse fell onto the padded floor and the time to fall was recorded and graphed.

### Study Results

To determine whether increased wheel activity was accompanied by an increase in muscle strength, near the end of the frailty study the mice were subjected to a grid hang test, which measures forearm grip strength. The mice were tested at 24 months and again at 26 months after 64 days of treatment with BGE-105 or vehicle. Average latency to fall increased in the BGE-105-treated mice (p=0.04, Mann-Whitney U test) (FIGS. 3B and 3C). Thus, the increase in running activity observed in BGE-105-treated mice were accompanied by improved forearm grip strength. At the end of the study, the BGE-105-treated mice had significantly higher tissue wet weight in the TA (FIG. 3F), and a trend toward higher tissue wet weight in the gastrocnemius and quadriceps (FIGS. 3G and 3H), and no difference in heart (FIG. 3I). The BGE-105-treated mice also had higher body weights (FIG. 3E), although the increase was not significant, just at p-value cutoff (0.0850).

### 6.3. Example 3: BGE-105 Activates Apelin Receptor Signaling Pathways

Administration of an apelin receptor agonist can induce the phosphorylation and activation of AMPK in heart tissue. Tissue samples were lysed using T-PER tissue protein extraction reagent (Thermo Fisher Scientific #78510) containing EDTA and protease/phosphatase inhibitors on the Omni Bead Ruptor 12 Homogenizer. Total protein was extracted then quantified using Pierce™ BCA Protein Assay Kit. Loaded equal amounts of total protein per lane on a 4-12% SDS-PAGE gel and transferred to PVDF membrane. Membranes were blocked and blotted with anti-phospho-AMPKα-Thr172 (Cell Signaling Technology, CST #2535), total-AMPKα (CST #2532), anti-phospho-Akt-SeR⁴⁷³ (CST #4060), total-Akt (CST #4685), anti-phospho-ERK-1/2-Thr202/Tyr204 of Erk1 and Thr185/Tyr187 of Erk2 (CST #4370), total-ERK-1/2 (CST #9107) or anti-APLNR receptor (abcam, ab214369) antibodies. Band intensities were normalized to loading control anti-β-Actin (CST #3700) or anti-GAPDH (abcam, ab181602) antibodies. Immunoreactive proteins were detected using SuperSignal™ West Femto Substrate (Thermo Fisher Scientific #34095) and quantified by Image Lab™ software (Bio-Rad Laboratories, Inc.).

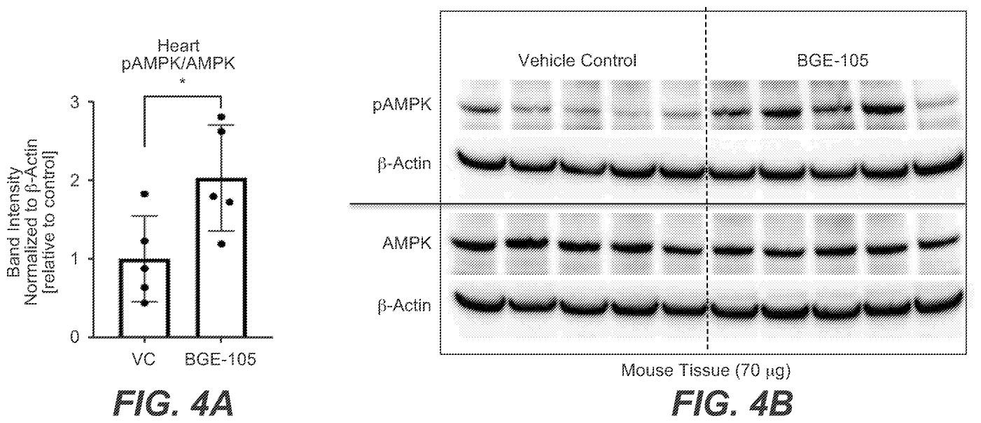

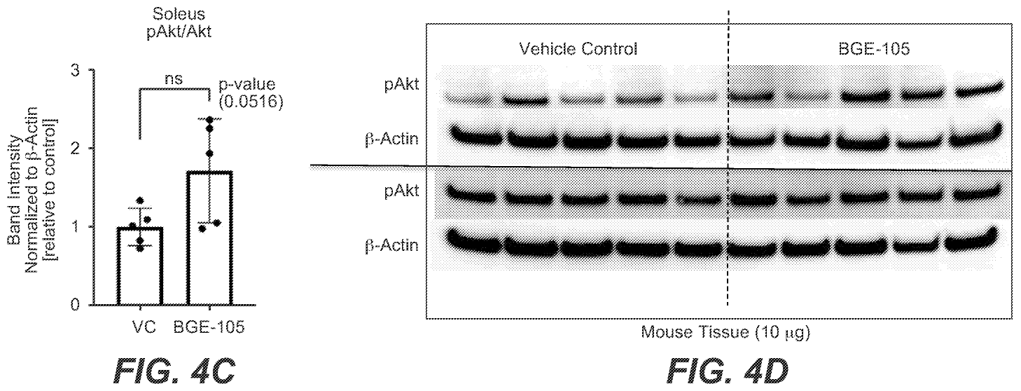

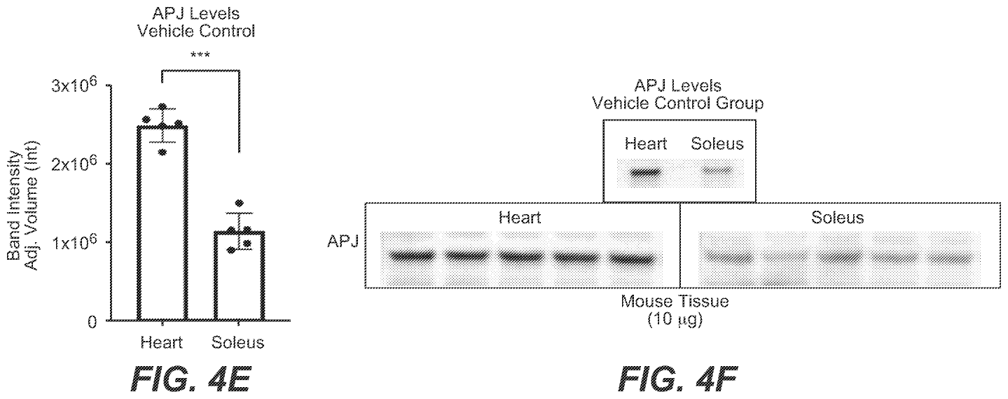

Following oral administration of 45 mg/kg BGE-105 or vehicle to mice, pAMPK levels in the heart were significantly higher in the BGE-105-treated group than in the vehicle control group, FIGS. 4A-4B. The effect of BGE-105 in skeletal muscle was also assessed. Apelin can induce the phosphorylation of Akt in the soleus muscle and improve glucose homeostasis. In the BGE-105-treated group, we observed a marginally significant increase in pAkt levels in the soleus, (p=0.0516), FIGS. 4C-4D. Per unit mass, the soleus contained about half as much apelin receptor as the heart, FIGS. 4E-4F, potentially explaining the stronger response in heart tissue.

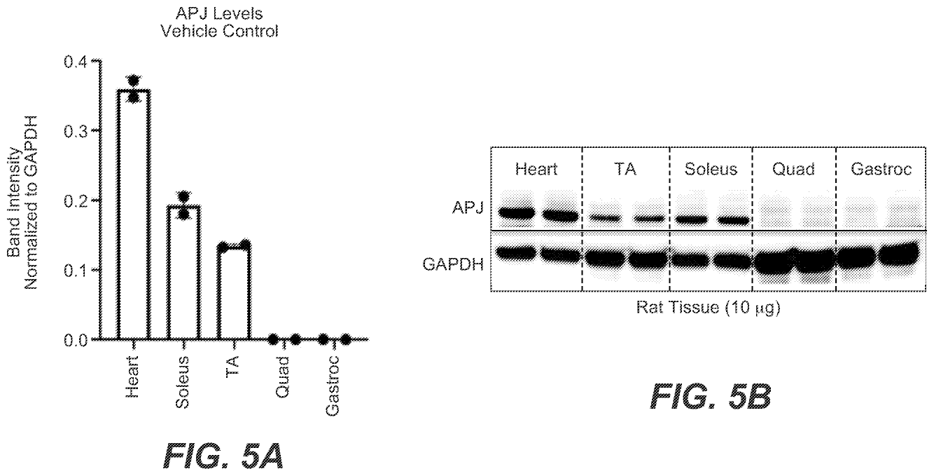

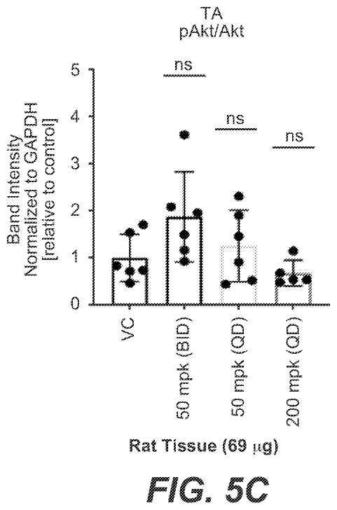

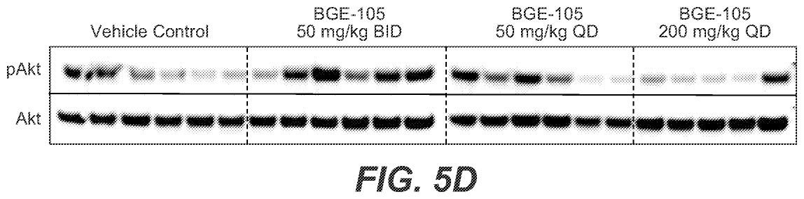

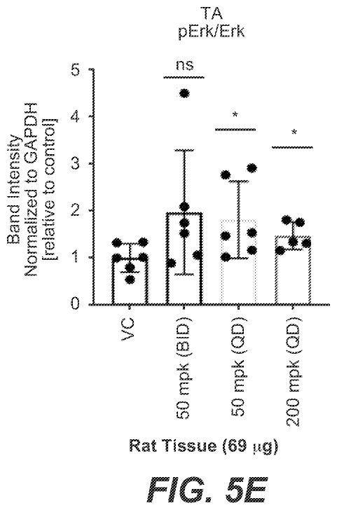

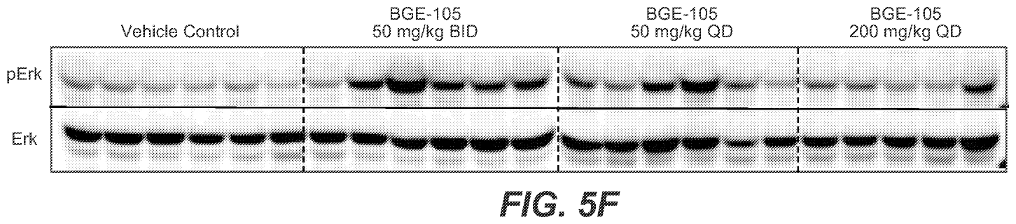

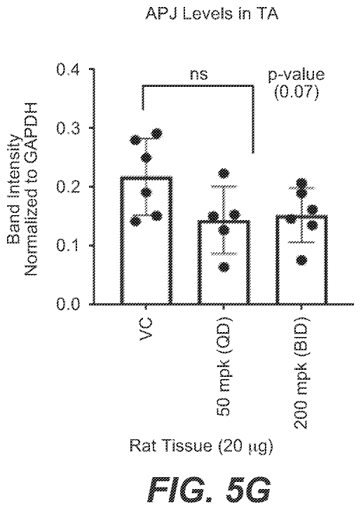

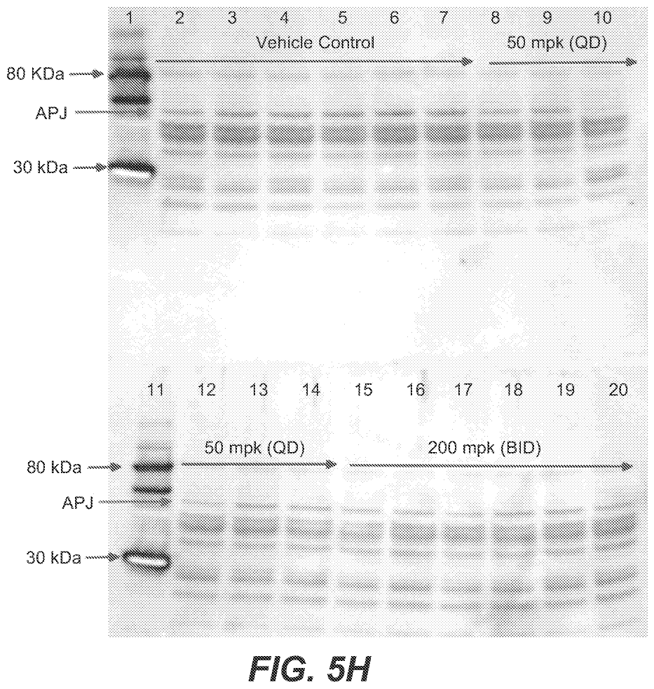

The difference between tissues was conserved among rodent species: In rats, as in mice, apelin receptor levels in rat tissue were 2-fold higher in heart than in soleus, FIGS. 5A-5B. Rat tibialis anterior (TA) had apelin receptor levels similar to those of soleus, and quadriceps and gastrocnemius had apelin receptor levels below the limit of detection in this western blot assay. Oral dosing of rats with BGE-105 for 5 consecutive days induced phosphorylation of Akt in TA in a dose-dependent manner, with 50 mg/kg BID eliciting the strongest response, FIGS. 5C-5D. A similar trend was observed for phosphorylation of Erk, FIGS. 5E-5F. Given that pErk is downstream of pAkt, this observation is consistent with a known signal transduction pathway. Next, we assessed the effect of chronic administration of BGE-105 on apelin receptor levels. BGE-105 elicited a modest but non-significant decrease in apelin receptor levels in the TA after 5 consecutive days of oral dosing at the lowest and highest doses tested, 50 mg/kg QD and 200 mg/kg BID respectively, FIGS. 5G-5I.

### 6.4. Example 4: BGE-105 Activates the Apelin Receptor in a Manner Similar to Apelin

We compared the abilities of BGE-105 and Pyr¹-Apelin-13 to activate APLNR receptor and recruit β-arrestin using the PathHunter β-arrestin assay.

The EC50 of BGE-105 was compared to Pyr¹-Apelin-13 on recruiting β-arrestin by either mouse or human APLNR using the PathHunter β-arrestin express GPCR Assay. APLNR activation was determined by β-arrestin recruitment as measured by the ProLink β-gal complementation technology (93-0001, DiscoveRx). In brief, CHO cells stably expressing APLNR were seeded and incubated overnight at 37° C. The compounds were tested in duplicate and diluted to obtain a 10-point curve with 3-fold serial dilutions (<1% DMSO). The compounds and cells were incubated for 3 hours at 37° C. After the incubation period the detection reagents were added and the plate chemiluminescent signal was measured after 30 min at RT.

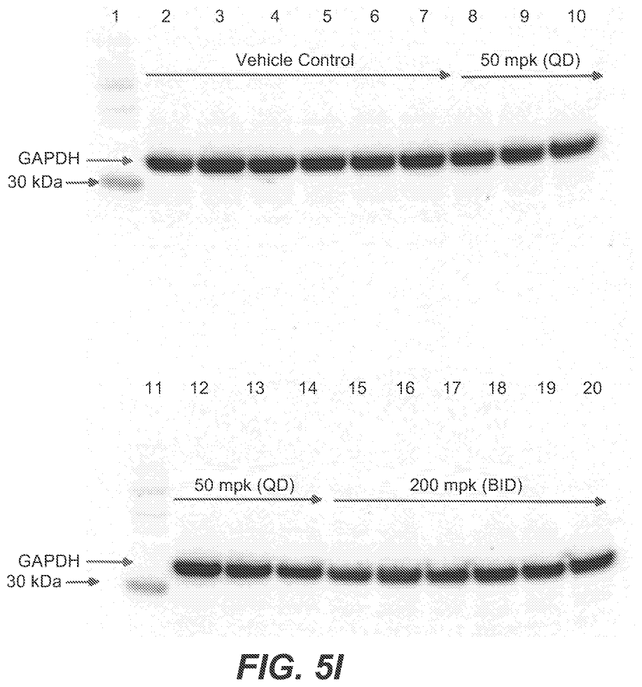

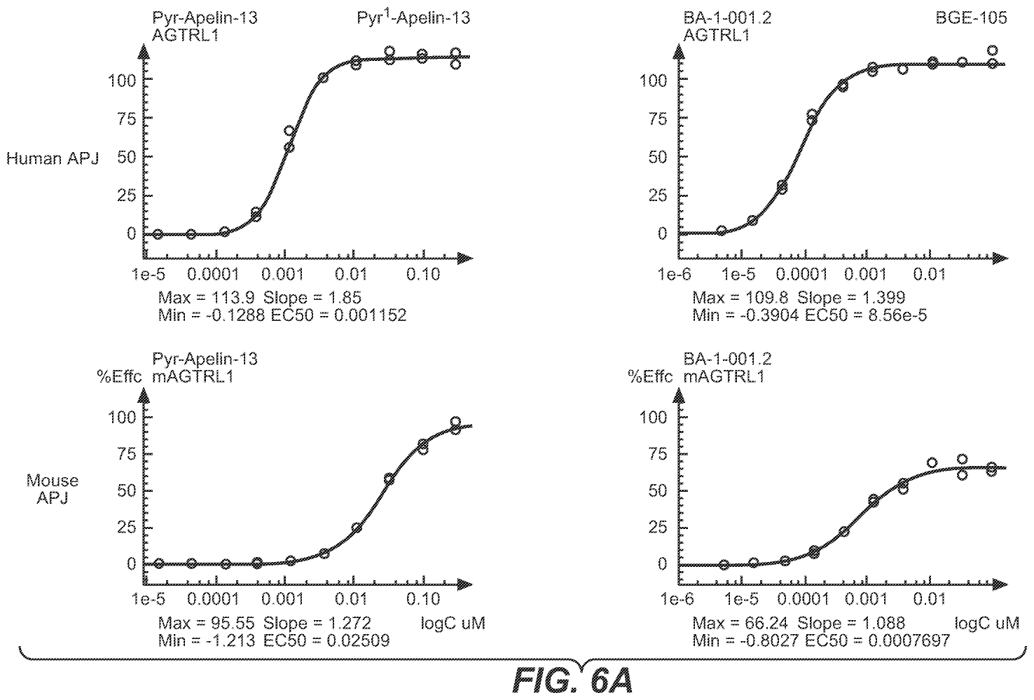

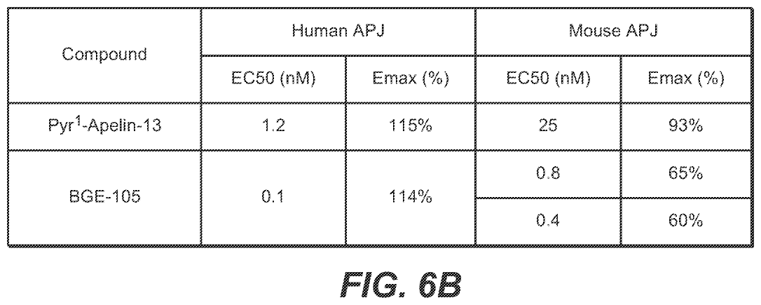

In cells stably expressing human APLNR, BGE-105 was 10-fold more potent than Pyr¹-Apelin-13: BGE-105, EC50=0.1 nM; Pyr¹-Apelin-13, EC50=1.2 nM, FIG. 6A. In cells stably expressing mouse APLNR, BGE-105 was 30-fold more potent than Pyr¹-Apelin-13: BGE-105, EC50=0.8 nM; Pyr¹-Apelin-13 (EC50=25 nM), FIG. 6A.

Although the increases in potency were comparable between human and mouse APLNR, the maximum effect (Emax) was not: human APLNR, Emax=114%; mouse APLNR, Emax=65%. Replication of the mouse APLNR β-arrestin assay with a fresh preparation of BGE-105 yielded similar data: Emax=60%, FIG. 6B. Thus, the potency of BGE-105 as an APLNR agonist may be higher in humans than in mice.

### 6.5. Example 5: BGE -105 Accelerates Regeneration of CTX-Induced Muscle Injury in Aged Mice

Impairment of muscle regeneration can contribute to age-related muscle weakness. This is particularly true in aged individuals who engage in physical activity. Exercise-induced muscle hypertrophy is linked to the capacity of muscle stem cells to be activated and promote regeneration. We evaluated the effects of oral treatment with BGE-105 during muscle regenerative processes (FIGS. 7A-7BB). To that end, we injected 18-month-old male (and 3-month old male) mice with cardiotoxin (CTX) in the left tibialis and gastrocnemius and then administered an oral bolus of BGE-105 (50 or 200 mg/kg/day) for 3 or 7 consecutive days.

Mice were i.p injected with buprenorphine (Centravet, 0.1 mg/kg) 30 minutes before injury and the day after. The day of the injury, mice were anesthetized with isoflurane inhalation and hindlimbs were shaved. Then, 10 μM of cardiotoxin (CTX, Latoxan, #L8102) was injected through two injections of 25 μl into the left tibialis muscle and two injections of 50 μl into the left gastrocnemius muscle, using a 22-gauge needle (Hamilton). Mice were euthanized 3 and 7 days after injury by cervical dislocation, muscles (PBS- and CTX-injected) were cut in two parts, one being snap frozen into liquid nitrogen for total RNA extraction and the other part being embedded into OCT, frozen in isopentane cooled with liquid nitrogen for histological analysis.

Mouse muscle samples were dissected and cryopreserved in OCT frozen in liquid nitrogen cooled isopentane. Samples were then sectioned at 10 μm on a cryostat and post-fixed with 4% Paraformaldehyde (PFA) for 15min at room temperature. Muscle frozen sections (10 μm) were stained by helaun/eosin or immune-labeled for laminin (Abcam) and embryonic myosin. Briefly, sections were blocked 1 h in PBS plus 4% BS(a), 2% goat serum, 0.01% Triton X-100. Sections were then incubated overnight with primary antibodies. After washes in PBS, sections were incubated 1 h with secondary antibodies anti-Ig2b AF 488 (Life Technology). Slides were finally mounted in ProLong Gold antifade Reagent (Molecular probes by Life Technology) with DAPI. Images were captured using a digital camera (Nanozoomer, Hamamatsu) attached to a motorized fluorescence microscope or using Olympus VS120 Virtual Microscopy Slide Scanning System. The area covered by eMHC-positive fibers and degenerated area was determined manually across the entire sections using the VS-ASW FL software measurement tools. The size of myofibers with central nuclei was calculated from laminin/DAPI staining on all fibers of the section and area determination were performed across the entire sections, using an automated image processing algorithm developed internally using the MetaXpress software (Molecular Devices).

### Study Results

The results presented in FIGS. 7A-7F demonstrate that, as previously demonstrated with apelin-13, BGE-105 treatment enhanced regeneration of tibialis 7 days after CTX injection. Indeed, the expression levels of genes involved in muscle regeneration (Pax7, MyoD, MyoG, Myf5, MyHC3, and MyHC8) were dramatically higher in BGE-105-treated animals than in their PBS-treated littermates. This effect was not as prominent in the gastrocnemius (FIGS. 7G-7L) suggesting that BGE-105 is more efficacious in muscle types with high receptor density, such as the tibialis. Moreover, BGE-105 was not as effective in young mice (FIGS. 7M-7X), suggesting that it is most efficacious in aged muscle with compromised repair capacity. Analysis of muscle size (cross-sectional area) confirmed the positive effect of BGE-105 on muscle regeneration in tibialis of aged mice (FIGS. 7Y-7Z) 3 and 7 days after CTX injection. Finally, BGE-105 treatment promoted accumulation of central nuclei in regenerating fibers (FIGS. 7AA-7BB), indicating that this compound can have a beneficial effect on regeneration.

Overall, the effect was less pronounced in the gastrocnemius, suggesting that an APJ agonist is most efficacious in tissues with high APJ receptor density e.g. tibialis anterior. It was also less effective in young mice suggesting that an APJ agonist is most efficacious in aged muscle with compromised repair capacity.

### 6.6. Example 6: BGE-105 Promotes Early Proliferation and Differentiation in Human Myoblast Cells

Immortalized human cells from male donors aged 25 years old (25-HMC) and 79 years old (79-HMC) are grown from the proliferation stage until they become 80% confluent, differentiate, and become myotubes. The cells were treated from day 1 to day 4 with either Pyr¹-Apelin-13 at 1 nM, BGE-105 at 0.05, 0.5, 5, 50 nM, or vehicle (<0.1% DMSO) (FIG. 8A to 8K). Early proliferation markers (Pax7, Myf5, MyoD, and MyoG were assessed via RT-PCR (FIG. 8D to 8K)

### Study Results

Short-term (from day 0 to day 4 post seeding) BGE-105 treatment induced a significant increase of cell proliferation in cells from both young and aged donors (FIGS. 8B-8C). BGE-105 treatment also increased the expression of muscle cell differentiation markers such as Pax7 and MyoD in young donor cells (FIGS. 8D-8G) and Pax7, Myf5, MyoD, and MyoG in aged-donor cells (FIGS. 8H-8K). Altogether, these results support the use of BGE-105 in human muscle physiology in aged populations.

### 6.7 Example 7: BGE-105 Prevents Disuse-Induced Muscle Atrophy in Aged Mice

BGE-105 activates pathways that benefit skeletal muscle physiology, notably the pAkt/pErk pathway, which plays a pivotal role in regulating muscle mass. Limb immobilization causes a loss of gross skeletal muscle mass accompanied by a significant decrease in apelin transcript levels. Hence, we tested whether BGE-105 rescues muscle atrophy induced by chronic immobilization. Because skeletal muscle atrophy caused by disuse is exaggerated during aging, we evaluated the effects of BGE-105 on maintenance of muscle mass in aged mice subjected to immobilization of the plantar flexor group (soleus, TA, EDL, gastrocnemius). Animals were orally administered vehicle or BGE-105 at 50 mg/kg BID; 1 week into treatment, the right hindlimb was immobilized by casting and the muscles were allowed to atrophy over 21 days.

Twenty-month-old male C57/B16 mice (n=10/group) were administered P.O. vehicle or BGE-105 at 50 mg/kg BID at ZT1 and ZT11.5. One week into the treatment, mice underwent modified hindlimb casting on one limb. Mice were anesthetized with isoflurane inhalation and the hindlimb wiped with povidone-iodine, then ethanol, and loosely wrapped in surgical gauze. A custom-made plastic immobilization device was placed on the limb, with the foot in full extension, so as to result in the maximal in vivo unloading of the plantarflexor group. The device was fixed to the hindlimb using Vetbond and the animal returned to its cage. After 3 weeks of treatment following casting, mice were euthanized 1 hour after the final ZT1 dose, and tissues isolated, weighed, then flash frozen in liquid nitrogen for subsequent western blot analysis.

### Study Results

Immobilization caused significant atrophy in the casted limb of the vehicle-treated group for all muscle types, FIG. 9A. Notably, wet weight of tibialis anterior (TA) muscle decreased by 25% in the placebo group, but by only 3% in the BGE-105 group, FIGS. 9D-9E. Similarly, extensor digitorum longus (EDL) atrophied by 14% in the placebo group but did not measurably atrophy in the BGE-105 group, FIGS. 9F-9G. A modest rescue in atrophy was observed in soleus, although this effect was not significant, FIGS. 9H-9I.

In these animals, gastrocnemius contained significantly less apelin receptor density than the other muscles, FIGS. 9J-9K, potentially explaining why BGE-105 is less effective in this muscle. FIGS. 9L-9M shows apelin receptor levels after one month of BGE-105 treatment at 50 mg/kg BID. Again, we observed that chronic activation of the apelin receptor by BGE-105 had a noticeable but non-significant drop in apelin receptor levels. Thus, although BGE-105 did downregulate the apelin receptor after chronic treatment, the effect was not significant at the dose tested.

Our data demonstrate that aged mice treated with BGE-105 were protected against some loss of muscle mass induced by immobilization. Thus, BGE-105 may have clinical benefits to protect against disuse atrophy in humans.

### 6.8. Example 8: BGE-105 Prevents Atrophy in Immobilized Human Muscles

Two groups of healthy older adult humans (e.g., N=10 per group) who are moderately active remain in bed continuously for 10 days, except for toileting, and they consume a eucaloric diet providing the recommended dietary allowance for protein (0.8 g/kg of protein per day). One group is given 200 mg of BGE-105 a day, while the other receives a placebo. Measurements before and after bed rest include muscle function and protein synthesis.

BGE-105 is shown to prevent or attenuate muscle atrophy in immobilized human muscles during periods of disuse.

What is claimed is:

1. A method of treating a muscle condition in a subject, the method comprising administering to a subject in need thereof an effective dose of an apelin receptor agonist of formula (I) or (II): 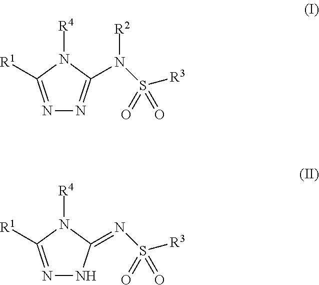
or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, O—(C₁-C₆ alkyl)—OH, —O—(C₁-C₆ alkyl)-O—(C₁-C₆ alkyl), —O—(C₁-C₆ haloalkyl)—OH, —O—(C₁-C₆ haloalkyl)-O—(C₁-C₆ alkyl), —O—(C₁-C₆ perhaloalkyl)—OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}—CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

2. The method of claim 1, wherein the muscle condition is an age-related muscle condition.

3. The method of any one of claims 1 to 2, wherein the subject is human and at least 40-years-old.

4. The method of claim 3, wherein the subject is at least 50-years-old.

5. The method of claim 4, wherein the subject is at least 60-years-old.

6. The method of claim 5, wherein the subject is at least 65-years-old.

7. The method of claim 6, wherein the subject is at least 70-years-old.

8. The method of claim 7, wherein the subject is at least 75-years-old.

9. The method of claim 8, wherein the subject is at least 80-years-old.

10. The method of any one of claims 1 to 9, wherein the muscle condition is a skeletal muscle condition.

11. The method of claim 10, wherein the skeletal muscle expresses the apelin receptor and administration of the apelin receptor agonist activates the apelin/APJ (APLNR) system in the muscle tissue of the subject.

12. The method of any one of claims 1 to 11, wherein the muscle condition is not a cardiovascular condition.

13. The method of any one of claims 1 to 11, wherein the subject is not suffering from, or at risk of, a heart failure.

14. The method of any one of claims 1 to 13, wherein the age-related muscle condition is associated with inflammation or impairment of mitochondrial function.

15. The method of any of one of claims 1 to 14, wherein the age-related muscle condition is associated with a loss-of-function of skeletal muscle, decrease in the ability to regenerate skeletal muscle, or decrease in the ability to heal after injury of skeletal muscle.

16. The method of any of one of claims 1 to 15, wherein the age-related muscle condition is associated with the loss-of-function of muscle stem cells.

17. The method of any one of claims 1 to 16, wherein the age-related muscle condition is selected from sarcopenia, frailty, hip fracture, ICU associated muscle weakness, mechanical ventilation-related muscle weakness, immobilization associated muscle weakness, recovery from muscle injury, muscle atrophy, diaphragm atrophy, and muscle wasting.

18. The method of any of one of claims 1 to 17, wherein the age-related muscle condition is associated with insulin insensitivity or Type 2 diabetes mellitus.

19. The method of any one of claims 1 to 18, wherein the human subject has, or is identified as having, low muscle strength.

20. The method of any one of claims 1 to 18, wherein the human subject has, or is identified as having, low muscle force.

21. The method of any one of claims 1 to 18, wherein the human subject has, or is identified as having, low lower limb muscle mass.

22. The method of any one of claims 1 to 18, wherein the human subject has, or is identified as having, low upper limb muscle mass.

23. The method of any one of claims 1 to 22, wherein the human subject has, or is identified as having, low muscle volume.

24. The method of claim 23, wherein the muscle volume is skeletal muscle volume.

25. The method of claim 24, wherein the muscle is tibialis anterior, tibialis posterior, gastrocnemius, sartorius, vastus intermedius, vastus laterals, vastus medialis, soleus, or extensor digitorum longus.

26. The method of any one of claims 1 to 25, wherein the apelin receptor agonist is administered orally, intravenously, intranasally, or intramuscularly.

27. The method of any one of claims 1 to 26, wherein the dose is administered daily.

28. The method of any one of claims 1 to 27, wherein the dose is administered as a plurality of equally or unequally divided sub-doses.

29. The method of any one of claims 1 to 28, wherein the dose is administered at varying dosing intervals.

30. The method of any one of claims 1 to 29, wherein the dose is 200 mg.

31. The method of claim one of claims 1 to 30, further comprising, assessing muscle mass after the dosing.

32. The method of claim 31, wherein the muscle mass is assessed at least one day after dosing.

33. The method of claim 32, wherein the muscle mass is assessed at least one week after dosing.

34. The method of claim 33, wherein the muscle mass is assessed at least one month after dosing.

35. The method of any of claims 1-34, wherein the subject has a low circulating level of apelin.

36. A method for maintaining and/or increasing muscle mass and/or muscle strength in an elderly subject, the method comprising administering to a subject in need thereof an effective dose of an apelin receptor agonist of formula (I) or (II): 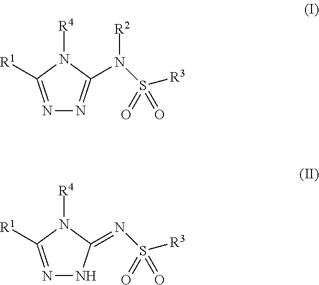
or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)—OH, —O—(C₁-C₆ alkyl)-O—(C₁-C₆ alkyl), —O—(C₁-C₆ haloalkyl)—OH, —O—(C₁-C₆ haloalkyl)-O—(C₁-C₆ alkyl), —O—(C₁-C₆ perhaloalkyl)—OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and —C₄ alkyl or is absent in the compounds of Formula II;
R³ is selected from an unsubstituted C₁-C₁₀ alkyl, a C₁-C₁₀ alkyl substituted with 1, 2, or 3 R^{3a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{3a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)—OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH,—C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

37. The method of claim 36, wherein the subject is at least 60-years-old.

38. The method of claim 37, wherein the subject is at least 65-years-old.

39. The method of claim 38, wherein the subject is at least 70-years-old.

40. The method of claim 39, wherein the subject is at least 75-years-old.

41. The method of claim 40, wherein the subject is at least 80-years-old.

42. The method of any one of claims 35 to 41, wherein the human subject has, or is identified as having, low muscle strength.

43. The method of any one of claims 35 to 42, wherein the human subject has, or is identified as having, low muscle force.

44. The method of any one of claims 35 to 43, wherein the human subject has, or is identified as having, low lower limb muscle mass.

45. The method of any one of claims 35 to 44, wherein the human subject has, or is identified as having, low upper limb muscle mass.

46. The method of any one of claims 35 to 45, wherein the human subject has, or is identified as having, low muscle volume.

47. The method of claim 46, wherein the muscle volume is skeletal muscle volume.

48. The method of claim 47, wherein the muscle is diaphragm, tibialis anterior, tibialis posterior, gastrocnemius, sartorius, vastus intermedius, vastus laterals, vastus medialis, soleus, or extensor digitorum longus.

49. The method of any one of claims 47 to 48, wherein the muscle is a skeletal muscle.

50. The method of any one of claims 35 to 49, wherein the human subject is mechanically ventilated.

51. The method of any one of claims 35 to 50, wherein the human subject has, or is identified as having reduced diaphragm thickness as compared to a human subject that is not mechanically ventilated.

52. The method of any one of claims 35 to 51, wherein the human subject has, or is identified as having diaphragm atrophy.

53. The method of any one of claims 35 to 51, wherein the human subject has, or is identified as having ventilator-induced diaphragmatic dysfunction (VIDD).

54. The method of any one of claims 35 to 53, wherein the human subject has, or is identified as having hypoxic respiratory failure.

55. The method of any one of claims 48 to 54, wherein the muscle expresses the apelin receptor.

56. The method of any of claims 35 to 55, wherein the human subject has a low circulating apelin level.

57. The method of any one of claims 35 to 56, wherein the apelin receptor agonist is administered orally, intravenously, intranasally, or intramuscularly.

58. The method of any one of claims 35 to 57, wherein the dose is administered daily.

59. The method of any one of claims 35 to 58, wherein the dose is administered as a plurality of equally or unequally divided sub-doses.

60. The method of any one of claims 35-59, wherein the dose is administrated intravenously.

61. The method of any one of claims 61 to 60, wherein the dose is administered for at least 1 hour.

62. The method of any one of claims 61 to 61, wherein the dose is administered for at least 20 hours.

63. The method of any one of claims 61 to 61, wherein the dose is administered for at least 22 hours.

64. The method of any one of claims 61 to 61, wherein the dose is administered for at least 100 hours.

65. The method of any one of claims 35 to 60, wherein the dose is at least 60 mg.

66. The method of any one of claims 35 to 60, wherein the dose is at least 120 mg.

67. The method of any one of claims 35 to 60, wherein the dose is at least 240 mg.

68. The method of any one of claims 35 to 60, wherein the dose is 200 mg.

69. The method of claim one of claims 35 to 60, further comprising, assessing muscle mass or muscle thickness after the dosing.

70. The method of claim 69, wherein the muscle mass is assessed at least one day after dosing.

71. The method of claim 70, wherein the muscle mass is assessed at least one week after dosing.

72. The method of claim 71, wherein the muscle mass is assessed at least one month after dosing.

73. The method of any one of claims 1 to 72, wherein R¹ is an unsubstituted pyridyl or is a pyridyl substituted with 1 or 2 R^{1a} substituents.

74. The method of any one of claims 1 to 73, wherein R^{1a} in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula 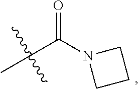 [SMILES: *C(=O)N1CCC1]
wherein the symbol [symbol] , when drawn across a bond, indicates the point of attachment to the rest of the molecule.

75. The method of any one of claims 1 to 74, wherein R¹ is selected from 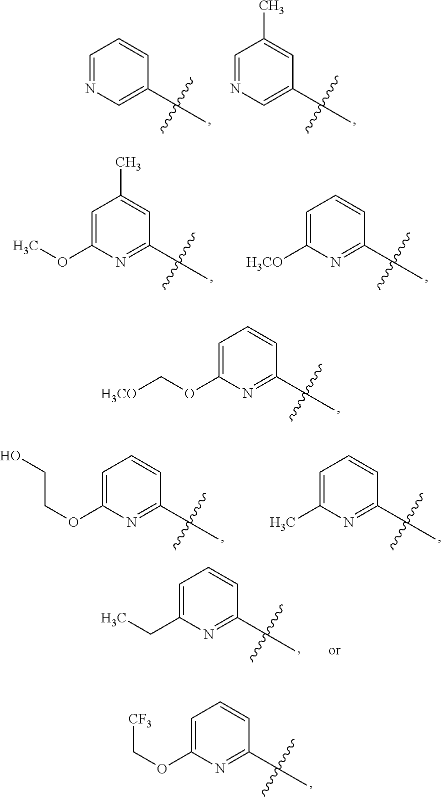
wherein the symbol [symbol] , when drawn across a bond, indicates the point of attachment to the rest of the molecule.

76. The method of any one of claims 1 to 75, wherein R² is —H.

77. The method of any one of claims 1 to 76, wherein R⁴ is a phenyl, pyridyl, pyrimidinyl, isoxazolyl, indolyl, naphthyl, or pyridinyl any of which may be unsubstituted or substituted with 1, 2, or 3 R^{4a} substituents.

78. The method of claim 77, wherein R⁴ is a phenyl substituted with 1 or 2 R^{4a} substituents.

79. The method of claim 78, wherein the 1 or 2 R^{4a} substituents are —O—(C₁-C₂ alkyl) groups.

80. The method of any one of claims 1 to 79, wherein R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

81. The method of any one of claims 1 to 80, wherein R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

82. The method of any one of claims 1 to 81, wherein Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

83. The method of any one of claims 1 to 82, wherein Q is a monocyclic heteroaryl group with 5 or 6 ring members containing 1 or 2 heteroatoms selected from N, O, or S and Q is unsubstituted or is substituted with 1 or 2 R^{Q} substituents.

84. The method of any one of claims 1 to 83, wherein R³ is a group of formula —(CR^{3d}R^{3e})—(CR^{3f}R^{3g})-Q.

85. The method of any one of claims 1 to 84, wherein R³ has the formula 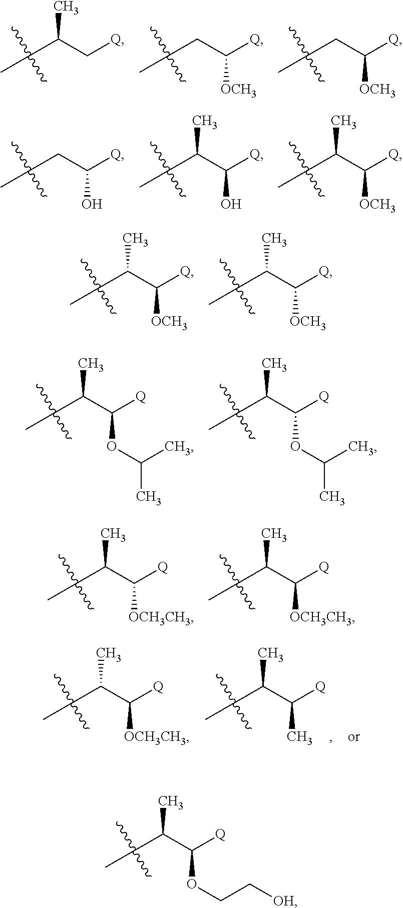
wherein the symbol [symbol] , when drawn across a bond, indicates the point of attachment to the rest of the molecule.

86. The method of any one of claims 1 to 85, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

87. The method of claim 86, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.
